(19) United States
(12) Patent Application Publication
(10) Pub. No.: US 2014/0017204 A1
(43) Pub. Date: Jan. 16, 2014

(54) Transfection of Mesothelium Body Cavity Lining with Gene Agents Followed by Chemotharapy to Treat Cancer of Organs in the Body Cavity

(71) Applicants: Nigel PARKER, Edgware (GB); Seppo YLA-HERTTUALA, Kuopio (FI)

(72) Inventors: Nigel PARKER, Edgware (GB); Seppo YLA-HERTTUALA, Kuopio (FI)

(73) Assignee: WKD Holding Oy, Kuopio (FI)

(21) Appl. No.: 14/030,399

(22) Filed: Sep. 18, 2013

Related U.S. Application Data
(63) Continuation of application No. 13/932,202, filed on Jul. 1, 2013.
(60) Provisional application No. 61/670,330, filed on Jul. 11, 2012.

(51) Int. Cl. A61K 38/21; A61K 31/555; A61K 45/06; A61K 39/395; A61K 31/337; A61K 31/415

(52) U.S. Cl. CPC A61K 38/212; A61K 31/337; A61K 31/415; A61K 45/06; A61K 39/3955; A61K 31/555
USPC 424/93.2

(57) ABSTRACT

Treating cancer of a organ located in a mesothelium-lined body cavity (i.e., lung, kidney, adrenal gland, ovary, prostate, pancreas or bladder cancer) by irrigating the mesothelium-lined body cavity with a solution containing a recombinant viral gene therapy vector bearing an interferon transgene, optionally administered shortly before administering chemotherapy and/or COX-2 inhibitor.

### RELATED APPLICATIONS

This application claims priority from pending U.S. patent application Ser. No. 13/932,202, filed on 1 Jul. 2013, which claims priority from U.S. Provisional Filing Ser. Nos. 61/670,330 filed 11 Jul. 2012, and 61/692,828 filed 24 Aug. 2012, the contents of which are incorporated here by reference.

### GOVERNMENT INTEREST

None

### BACKGROUND

Cancer of the body cavity organs is quite prevalent. For example, in 2008, there were 1.61 million new cases of lung cancer, and 1.38 million deaths due to lung cancer world-wide. By the time it presents itself clinically, organ cancer has frequently progressed to other local areas or metastasized to different parts of the body. Ovarian cancer similarly typically presents itself clinically only after reaching an advanced stage. See FIG. 1. Such late stage cancer presents a difficult target for gene therapy based therapeutic approaches.

As an example, malignant pleural mesothelioma is a form of lung cancer that affects the outer regions of the lungs, and spreads to invade other adjacent organs. Incidence in the United States is about 3,000 cases per year. While malignant pleural mesothelioma is known as a fatal cancer, the disease related morbidity and mortality is related not to metastases per se, but to the local invasion of vital structures like the chest wall and diaphragm.

As recently as 1990, malignant pleural mesothelioma was refractory to all known standard cancer therapies. Average four-year survival was not increased with surgery nor radiotherapy, and was decreased with then-standard cytotoxic chemotherapeutic agents.

In the early 2000s, pemetrexed was found to improve mesothelioma outcomes. While statistically significant, the improvement was not particularly great: pemetrexed combined with cisplatin increased median survival time from about 9 months to 12 months. The combination showed only about a 60% “disease control” rate (that is, the proportion of patients with stable or partially-responsive disease after treatment).

We have thus found a way to treat cancer of organs in mesothelium-lined body cavities (i.e., lung, kidney, ovary, prostate, adrenal gland, pancreas) by (a) irrigating the body cavity lining with a solution containing a recombinant viral gene therapy vector bearing a “homomimetic” transgene (that is, a transgene which codes for a polypeptide which mimics an effect of a naturally-occurring human polypeptide; examples of homomimetic transgenes include transgenes coding for interferon and transgenes coding for a human cell surface receptor agonist such as an agonist of a vascular endothelial growth factor receptor); and (b) administering cytotoxic chemotherapy, and (c) optionally administering COX-2 inhibitor. We have found that combining viral gene therapy treatment using a homomimetic transgene for interferon in advance of standard chemotherapy agents dramatically improves the efficacy of that chemotherapy. We have also found that administering viral gene therapy as a body cavity irrigation (rather than as an injection into the tumor or cancerous organ) dramatically improves the efficacy of later administered chemotherapy.

### BRIEF DESCRIPTION OF THE DRAWINGS

FIG. 1 is a photograph of ovarian cancer matured to a stage it could typically be identified clinically.

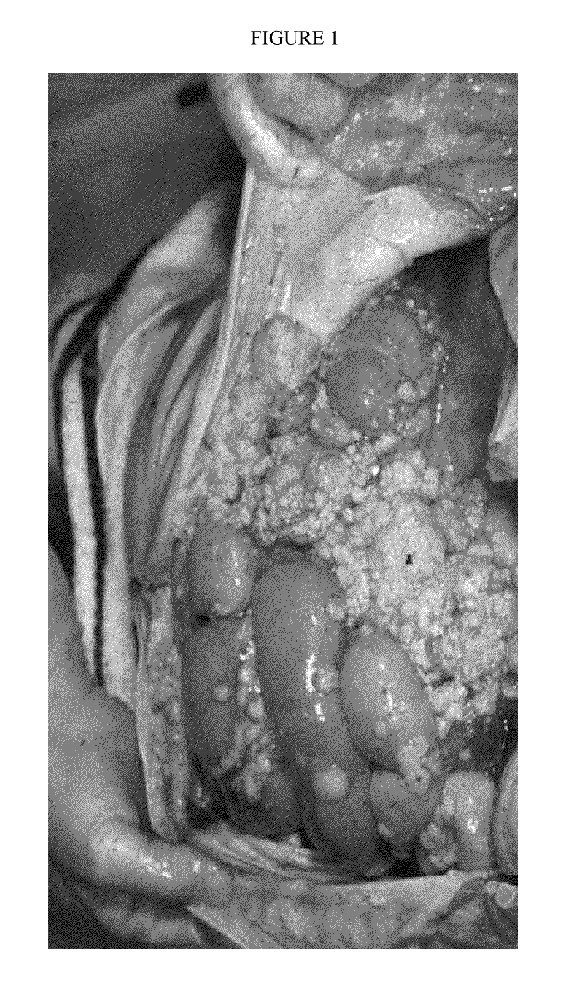

FIG. 2, Mesothelioma Ad. tk Gene Rx Schema, schematically illustrates our method of irrigating a solution containing gene therapy vector into the mesothelium-lined cavity surrounding the lungs using a catheter.

FIG. 3 schematically illustrates the ovary located inside a mesothelium-lined body cavity.

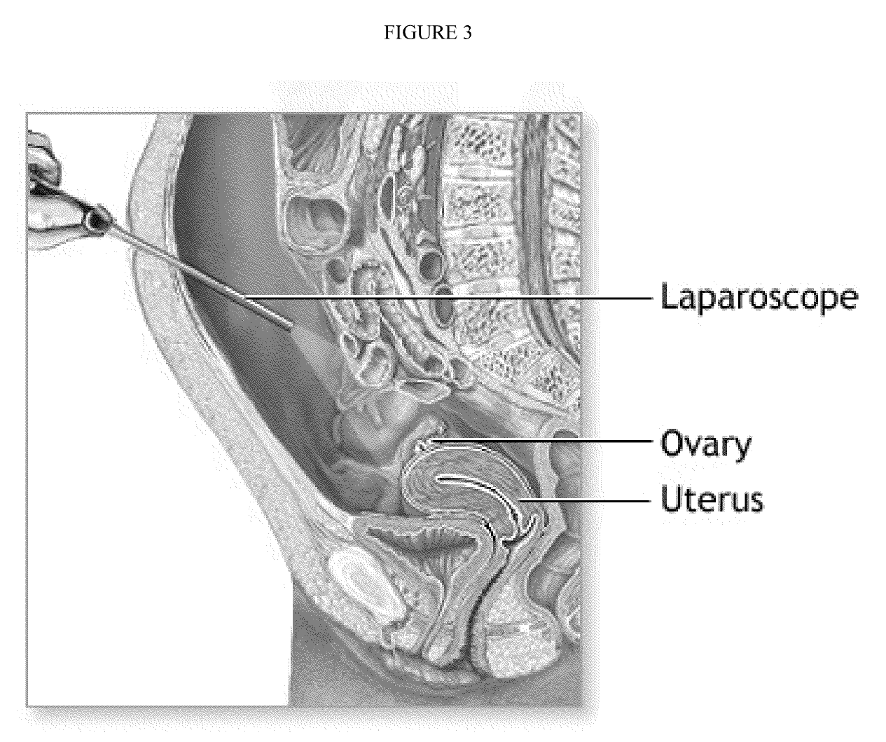

FIG. 4 is a chart showing the daily level of interferon expression of a gene therapy-administered interferon beta transgene, measured by nanograms of interferon per mL of patient blood serum, with two doses of vector (t=0 and t=15 days) (n=8 patients).

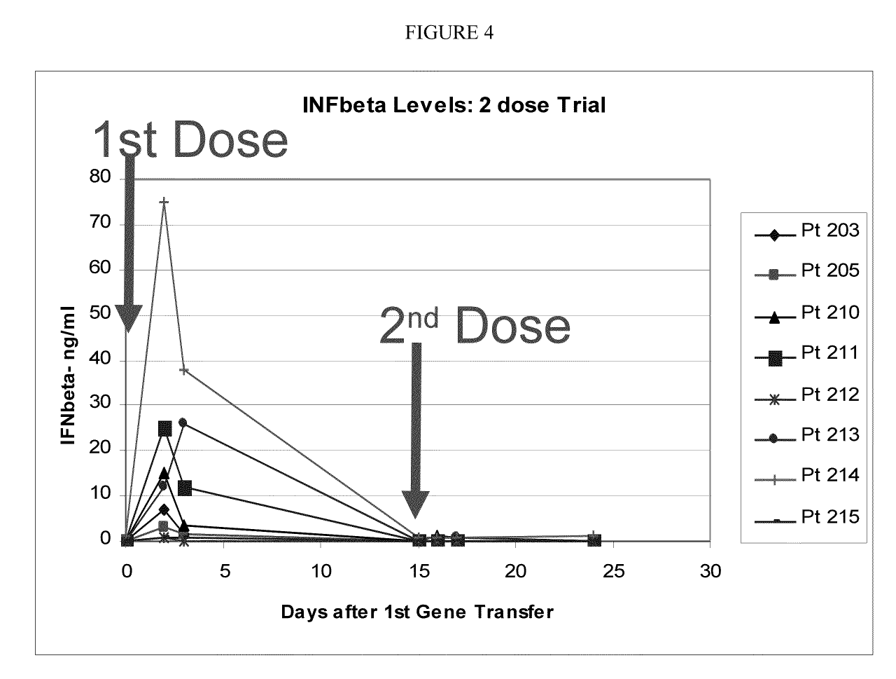

FIG. 5 is a chart showing the daily level of interferon expression of a gene therapy-administered interferon beta transgene, measured by nanograms of interferon per mL of patient blood serum, with two doses of vector (t=0 and t=8 days) (n=4 patients).

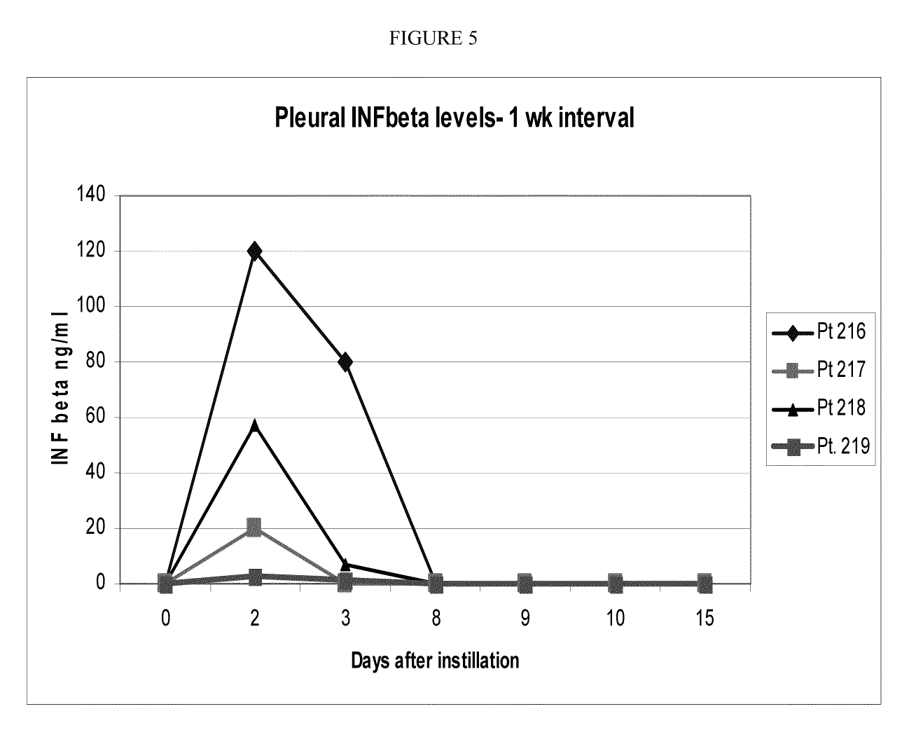

FIG. 6 is a chart showing titer of virus-neutralizing antibody over time in virus-treated patients.

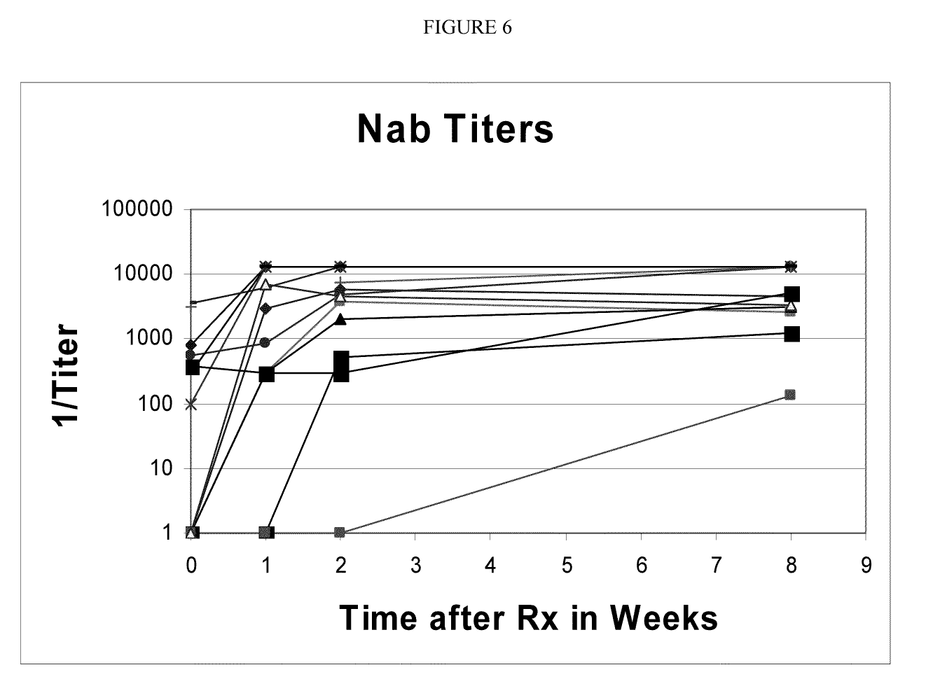

FIG. 7 shows increasing neutralizing antibody titer correlates with lower levels of interferon beta expression.

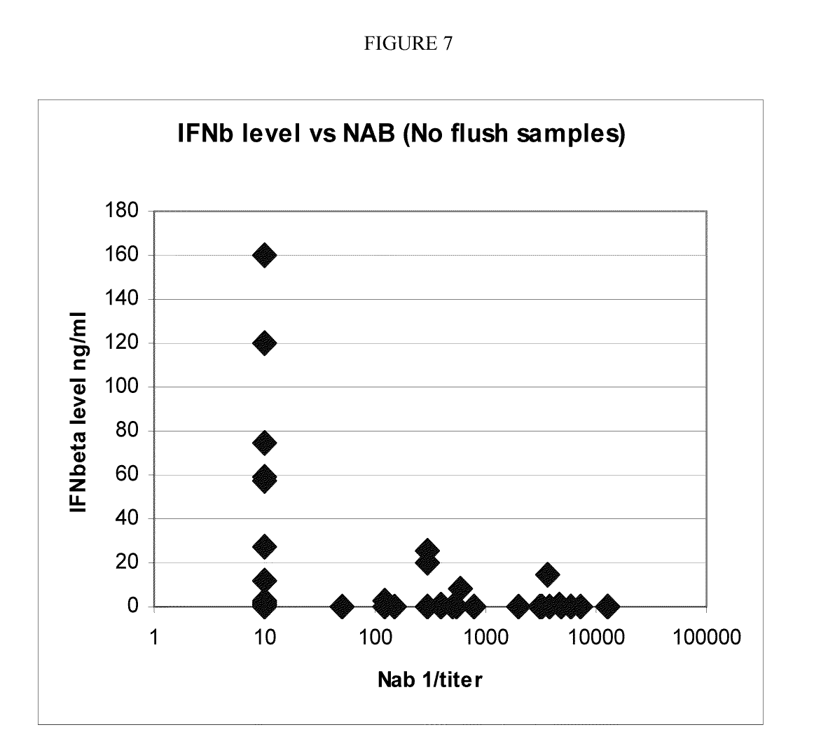

FIG. 8 shows PAGE immunoblots for patient Nos. 107, 112 and 106, before and after vector treatment, showing vector treatment induces production of antibodies specific for cancer antigen.

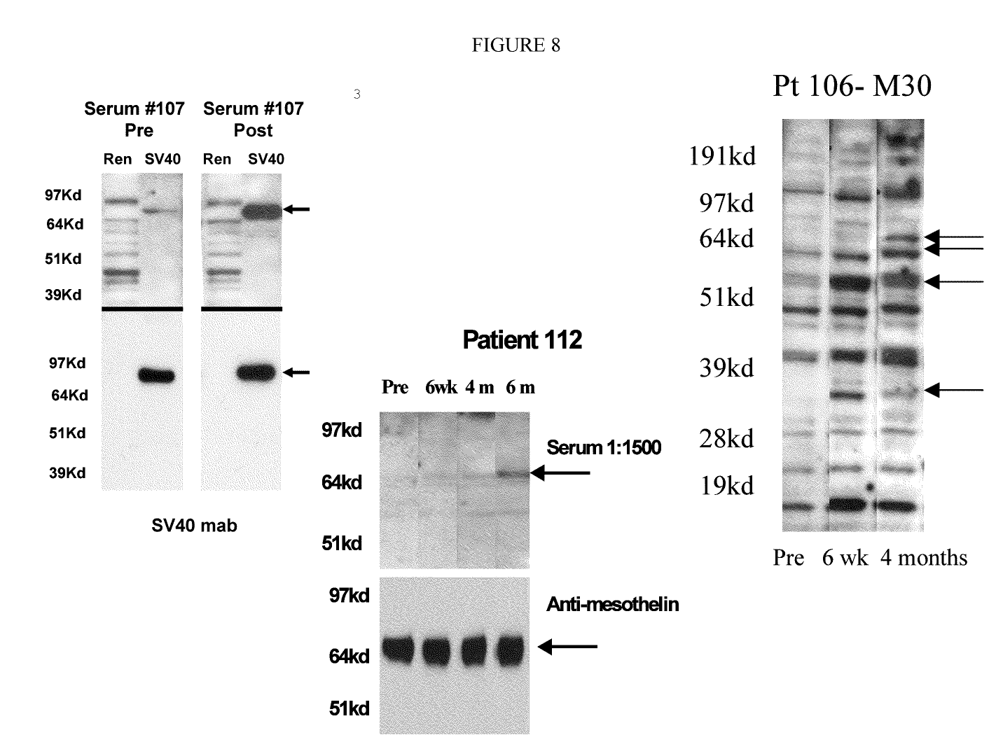

FIG. 9 is a chart of results for ten human patients, each with cancer of mesothelial-derived body cavity organs (mesothelioma, lung cancer or ovarian cancer), administered one dose of recombinant adenovirus bearing an interferon beta transgene.

FIG. 10 is a chart of results for human patients (n=13), each with cancer of an organ in a mesothelium-lined body cavity: mesothelioma, other lung cancer, “pleural adeno” cancer (i.e., lung cancer originating in the lung which has grown distally to affect the pleural membrane), “lung adeno” cancer or breast cancer. Each patient was administered two doses of recombinant adenovirus bearing an interferon beta transgene at a two-week interval.

FIG. 11 is a chart of results for human patients (n=4), each with cancer of an organ in a mesothelium-lined body cavity (mesothelioma, breast cancer or ovarian cancer), administered two doses of recombinant adenovirus bearing an interferon beta transgene at a one week interval.

FIG. 12 is a mesothelioma lung cancer survival curve for the prior art cisplatin monotherapy (n=222) and combination cisplatin+pemetrexed therapy (n=226).

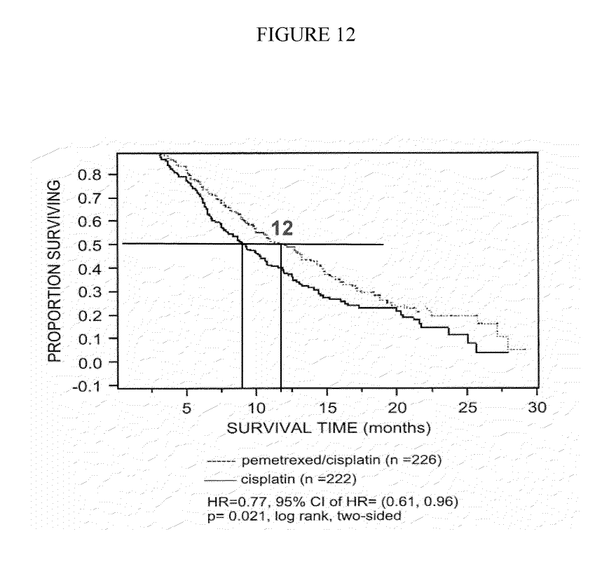

FIG. 13 is a mesothelioma lung cancer survival curve for recombinant adenoviral vector bearing an interferon beta transgene (n=17).

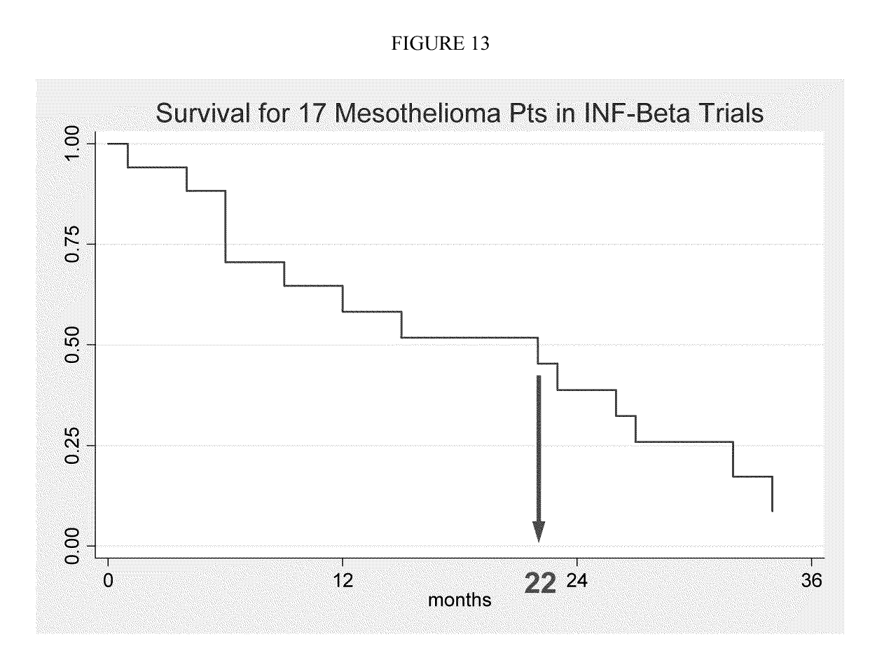

FIG. 14 is a pair of PAGE immunoblots for patient #301, showing inducement of antibodies specific against the circa-30 kD osteopontin mesotheliomal lung cell line antigen.

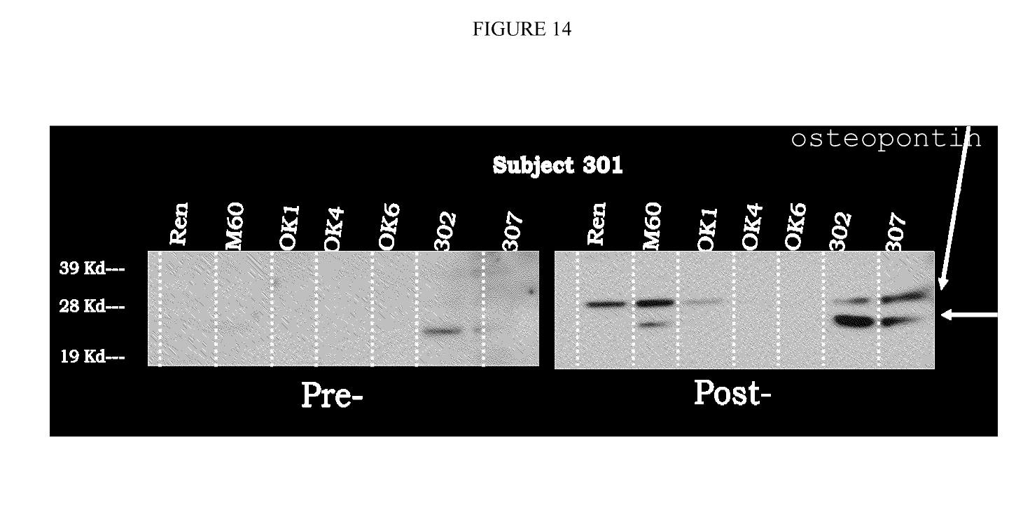

FIG. 15 shows recombinant adenoviral vector with an interferon beta transgene induces systemic NK cells.

FIG. 16 is a table of clinical outcomes of patients (n=9) administered two sequential doses of recombinant adenoviral vector with an interferon alpha transgene.

FIG. 17 shows tomography scans of the thoracic cavity of Patient #309 pre-therapy, two months post adenoviral therapy and six months post therapy.

FIG. 18 compares the tumor volume 30 days after tumor cell implantation of murine lung cancer cell line flank tumors injected intra-tumorally with recombinant adenovirus bearing an interferon beta transgene, where the treatment was given when the average tumor volume was 126 mm³ (day 4), or 439 mm³ (day 7) or 929 mm³ (day 13), showing that larger tumors are more resistant to gene therapy treatment.

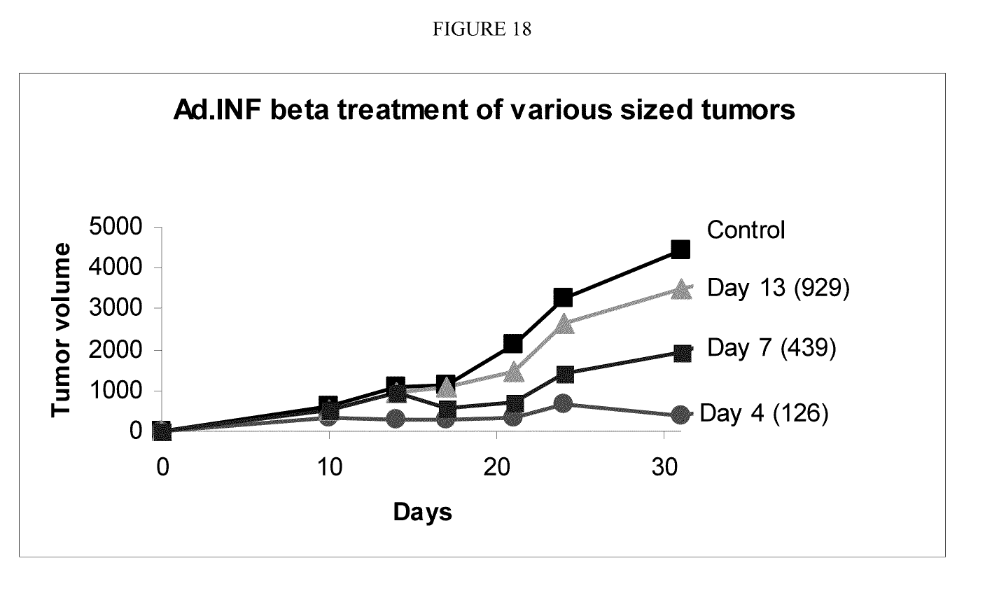

FIG. 19 charts the size of implanted murine lung cancer cell line flank tumors treated with recombinant adenovirus bearing an interferon alpha transgene or treated with chemotherapy or treated with both recombinant adenovirus and chemotherapy.

FIG. 20 is a bar chart comparing murine tumor volume, 23 days after tumor cell implant, of control; pemetrexed+cisplatin; interferon beta gene therapy vector; pemetrexed+cisplatin given two days before interferon beta gene therapy vector; and pemetrexed+cisplatin given two days after interferon beta gene therapy vector.

FIG. 21 is a chart comparing murine tumor volume of control; gemcitabine (administered day=0); interferon beta gene therapy vector (administered day=0); and interferon beta gene therapy vector (day=0) followed by gemcitabine (day=3).

FIG. 22 is a chart comparing murine tumor volume treated with celecoxib, a COX-2 inhibitor, days 14 to 27 after tumor cell implant; interferon alpha gene therapy vector (day=17); cisplatin+gemcitabine (day=20 and 27); and COX-2, interferon alpha gene therapy vector, and cisplatin+gemcitabine.

FIG. 23 is a schematic diagram of our treatment protocol used in our Phase I/II human clinical trial of COX-2, interferon alpha gene therapy vector, and chemotherapy (pemetrexed with cisplatin, pemetrexed with carboplatin, gemcitabine alone, or gemcitabine with carboplatin).

FIG. 24 is a Table showing clinical results for human patients (n=12) treated according to the protocol of FIG. 23.

FIG. 25 is a tomography scan of the chest cavity of Patient #405, before and after treatment according to the protocol of FIG. 23.

FIG. 26 is a tomography scan of the chest cavity of Patient #406, before and after treatment according to the protocol of FIG. 23.

FIG. 27 is a tomography scan of the chest cavity of Patient #406, before and after treatment according to the protocol of FIG. 23.

FIG. 28 is a tomography scan of the chest cavity of the progressive disease of Patient #407 (a non-responder), before and after treatment according to the protocol of FIG. 23.

FIG. 29 is a tomography scan of the chest cavity of Patient #408, before and after treatment according to the protocol of FIG. 23.

FIG. 30 is a table showing the characterization, MRI-volumes and tumor weights of the study group at the end of the follow-up (mean±SEM).

FIG. 31 is a time line showing the protocol of the study.

FIG. 32 is an MRI measurement of tumor growth and tumor weights after 1 week of treatment.

FIG. 33 is an MRI measurement of tumor growth and tumor weights at the end of the follow up.

FIG. 34 is MRI images of the growth of the ovarian tumors in control, gene therapy and becacizumab-treated mouse before (MRI I), 1 week (MRI II) and 2 weeks (MRI III) after the treatment. Tumors are marked with arrows and circled. MRI<magnetic resonance imaging. *p<0.05; **p<0.001; ***p<0.001.

FIG. 35 is a histology and measurements of the intraperitoneal ovarian tumors Hematoxylin-eosin staining of serous cystadenocarcinoma in control group.

FIG. 36 is a histology and measurements of the intraperitoneal ovarian tumors CD-34-positive microvessels in tumor tissues of different treatment groups.

FIG. 37 histology and measurements of the intraperitoneal ovarian tumors Microvessel density (MVD, microvessels/mm²) was significantly reduced in mice treated with gene therapy alone (p=0.001) and together with paclitaxel (p=0.008). Gene therapy significantly reduced the total area of tumors covered by microvessels (p=0.0005; TVA, tumor vascular area). *p<0.05; **p<0.001; ***p<0.001. Magnification, ×100; bar, 100 μm. Error bars, SEM.

FIG. 38 shows Kaplan-Meier survival analysis curves.

FIG. 39 is a table showing the clinical chemistry after the treatment.

### DETAILED DESCRIPTION

Support for the instant claims is provided by pre-clinical research on cancer of the ovaries, and by pre-clinical and clinical research on cancer of the ovaries, breast and lung. We discuss each in turn.

Ovarian Cancer

We compared effects of antiangiogenic gene therapy with a combination of soluble sVEGFR-1, sVEGFR-2 and sVEGFR-3 to chemotherapy with carboplatin and paclitaxel and to antiangiogenic monoclonal anti-VEGF-antibody bevacizumab in an intraperitoneal ovarian cancer xenograft model in mice (n=80). Gene therapy was also combined with chemotherapy. Therapy was initiated when sizable tumors were confirmed in magnetic resonance imaging (MRI). Adenovirus-mediated gene transfer was performed intravenously (2×109 pfu), while chemotherapy and monoclonal anti-VEGF-antibody were dosed intraperitoneally. The study groups were as follows: AdLacZ control (n=21); combination of AdsVEGFR-1, -2 and -3 (n=21); combination of AdsVEGFR-1, -2, -3 and paclitaxel (n=9); bevacizumab (n=14); paclitaxel (n=9) and carboplatin (n=5). Effectiveness was assessed by survival time and surrogate measures such as sequential MRI, immunohistochemistry, microvessel density and tumor growth. Antiangiogenic gene therapy combined with paclitaxel significantly prolonged the mean survival of mice (25 days) compared to the controls (15 days) and all other treatment groups (p=0.001). Bevacizumab treatment did not have any significant effect on the survival. Tumors of the mice treated by gene therapy were significantly smaller than in the control group (p=0.021). The mean vascular density and total vascular area were also significantly smaller in the tumors of the gene therapy group (p=0.01). These results show potential of the antiangiogenic gene therapy to improve efficacy of chemotherapy with paclitaxel and support testing of this approach in a phase I clinical trial for the treatment of ovarian cancer.

Material and Methods

Cell line: SKOV-3m cell line has been characterized earlier. Cells were cultured in McCoy's 5A medium (Gibco, Invitrogen, Life Technologies). Cancer cells were tested to be mycoplasma free. Before in vivo inoculation, the cells were trypsinized and counted.

Chemotherapy and anti-VEGF-antibody: Carboplatin 10 mg/ml infusion concentrate was used at a dose of 80 mg/kg for the primary treatment of Balb/cA-nu mice. The dose per Balb/cA-nu was 1.6 mg/500 μl NaCl 0.9%. Paclitaxel 6 mg/ml concentrate was used at a dose of 20 mg/kg. The dose per nude mouse was 320 μg/500 μl NaCl 0.9%. Bevacizumab 25 mg/ml concentrate was used at a dose of 5 mg/kg, and the dose per nude mouse was 100 μg/500 μl NaCl 0.9%. It was injected every fifth day.

Viral vectors: Adenoviral vectors encoding sVEGFR-1-IgG fusion protein, sVEGFR-2-IgG fusion protein, sVEGFR-3-IgG fusion protein and LacZ (AdLacZ) as a control vector were used for the study. Replication-deficient E1-E3-deleted clinical GMP-grade adenoviruses were produced in 293 cells. Adenoviruses were analyzed to be free from helper viruses, lipopolysaccharides and bacteriological contaminants.

Animal model: Eight to 10-weeks-old (n ¼ 80) Balb/cA-nu female nude mice were used for the studies. Ovarian carcinoma was produced by inoculating 1×10⁷ SKOV-3m cells into the peritoneal cavity of nude mice with a 22 G needle. Development of the ovarian cancerous tumors was followed by sequential MRI. When the first solid, measurable tumor was detected in MRI, gene transfer or other treatment was started on the following day. Mice were randomly divided into six groups:

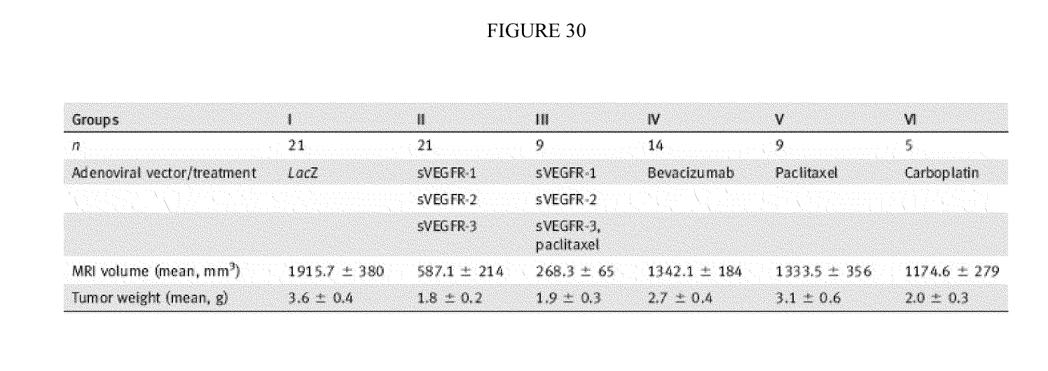

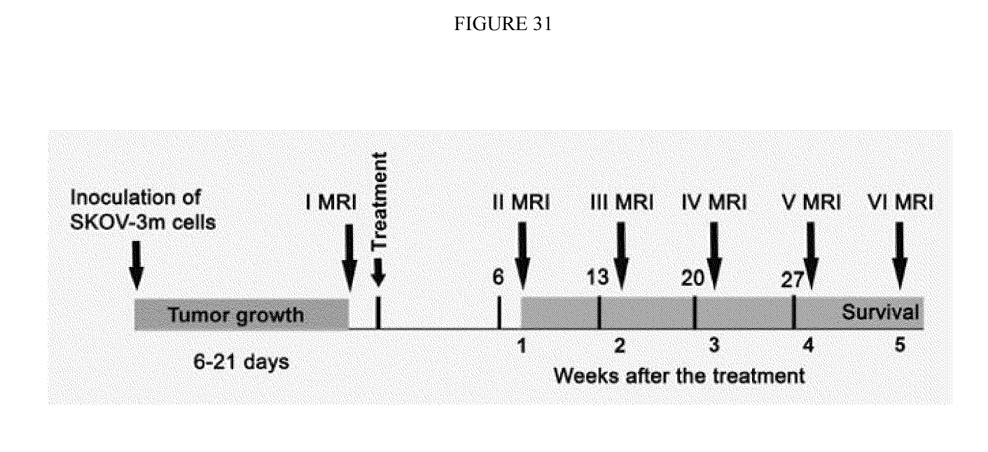

21 animals received combination of AdsVEGFR-1, AdsVEGFR-2 and AdsVEGFR-3 (2×10⁹ pfu/200 μl) once; 14 animals received bevacizumab 100 μg/500 μl every fifth day until sacrifice; nine animals received paclitaxel 320 μg/500 μl once as a single therapy and five animals received carboplatin 1.6 mg/500 μl once as a single treatment; nine animals received a combination of AdsVEGFR-1, AdsVEGFR-2 and AdsVEGFR-3 (2×10⁹ pfu/200 μl) once, and after 1 week paclitaxel 320 μg/500 μl as a one shot; 21 control animals received AdLacZ (2×10⁹ pfu; FIG. 30 and FIG. 31). Tumors developed within 3 weeks after the inoculation of the tumor cells. The presence of all tumors was verified by MRI (magnetic resonance imaging) before starting the therapy. Tumors were observed weekly until the death of the mice. Plasma samples were collected 6, 13, 20 and 27 days after the treatment. Gene transfer was performed intravenously (i.v.) via tail vein in the final volume of 200 ll in 0.9% saline. Paclitaxel, bevacizumab and carboplatin were dosed intraperitoneally (i.p.).

MRI was done weekly after gene transfer or treatment, and tumor volumes were measured. The overall follow-up time lasted until the appearance of significant symptoms necessitating sacrifice or to the death. At the time of death, all tumor tissue, liver, spleen, kidneys and lungs were harvested, and tumor masses were weighed. Ascites fluid was collected in a syringe. The mice were kept in a pathogen-free isolated unit at the National Experimental Animal Center of the University of Eastern Finland. Food, water and sawdust bedding were autoclaved, and the mice received chow and water ad libitum. All animal studies were accepted by the Experimental Animal Committee of the University of Eastern Finland.

Histology, Immunohistochemistry, Microvessel Measurements and Real-Time Quantitative PCR:

Tissue samples were immersed in 4% paraformaldehyde for 4-6 h, followed by overnight immersion in 15% sucrose. The specimens were embedded in paraffin, and 5-l m thick sections were processed for hematoxylin-eosin, Ki-67 (DakoCytomation, Glostrup, Denmark), CD-34 (HyCult biotechnology b.v., AA Uden, The Netherlands) and LYVE-1 (ReliaTech GmbH, Braunschweig, Germany) stainings.

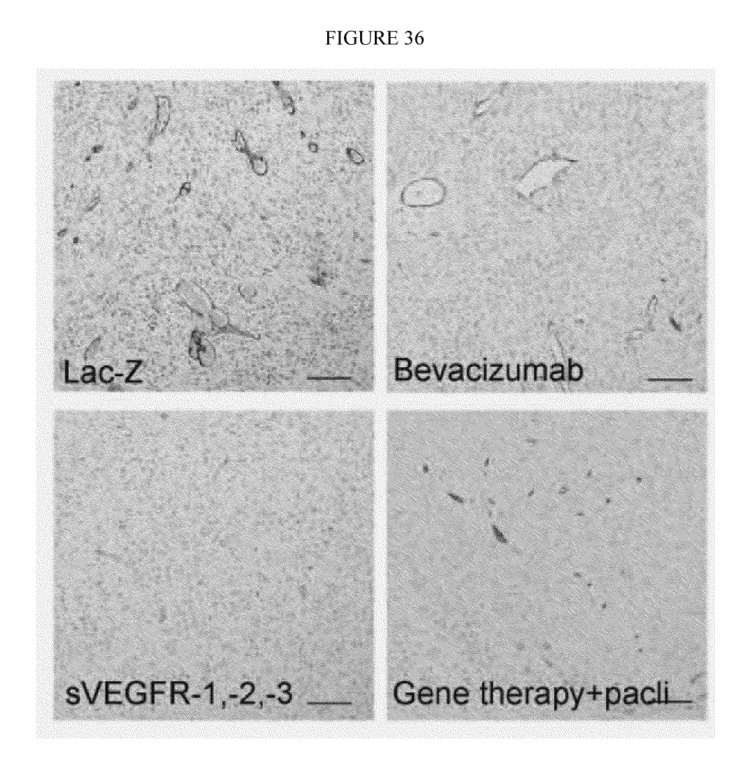

Photographs of histological sections were taken and processed using an Olympus AX70 microscope (Olympus Optical, Japan) and analySIS (Soft Imaging System, GmbH, Germany) and PhotoShop (Adobe) softwares. Microvessel density (MVD) and total microvascular area (%) of the tumors (TVA) were measured from CD34-immunostained sections using analySIS software at 100× magnification in a blinded manner (FIG. 36). Six to 10 different fields, which represented maximum MVD areas, were selected from each tumor. Necrotic areas were avoided. In addition, the total number of LYVE-1-positive lymphatic vessels per section was counted. Mean 6 SEM of the measurements are reported.

Gene expression levels of human and mouse VEGF-A, VEGF-B, VEGF-C, VEGF-D and PLGF in SKOV-3m cells and tumors from AdLacZ-injected mice were determined with real-time quantitative PCR (StepOnePlus instrument and software, Applied Biosystems) with gene expression assays (TaqMan-chemistry based probes, Applied Biosystems).

Magnetic Resonance Imaging:

To follow the development of ovarian carcinoma and to measure tumor volumes, MRI was performed using a 9.4 T vertical magnet (Oxford Instruments, Oxford, UK) equipped with actively shielded field gradients (Magnex Scientific, Abdington, UK) interfaced to a Varian DirectDrive console (Varian, Palo Alto, Calif.).

Mice were anesthetized with an s.c. injection of a mixture of fentanyl-fluanisone (Jansen Pharmaceutica, Hypnorm, Buckinghamshire, UK) and midazolame (Roche, Dormicum 5 mg/ml, Espoo, Finland). For a signal transmission and reception, a mouse body surface coil (m2m Imaging Corp., Cleveland, Ohio) was used. Axial T2-weighted images were acquired (repetition time=2.5 s, echo time=11 ms, field of view=35×35 mm², resolution=256×128, slice thickness=1 mm and number of slices=25). Tumor volumes were measured manually (MatLab, Math-Works, Natick, Mass.). The tumor masses differed from surrounding nontumor soft tissue with intensity and location. To measure tumor volume (mm³), area of the tumor (mm²) was calculated from each slide and then multiplied with the summation of the areas by the slice thickness. If more than one tumor nodule was detected from the MRI scan, the tumor volume was taken as a sum of all nodules. MRI was performed weekly after the first tumors were detectable.

Clinical Chemistry:

Plasma samples were collected at 6, 13, 20 and 27 days after the gene transfer, and when the mice were sacrificed. Alanine aminotransferase (ALT) and creatinine (Crea) were monitored using routine clinical chemistry assays at Kuopio University Hospital Central Laboratory. Enzyme-linked immunosorbent assays (Quantikine; R&D Systems, Minneapolis, Minn.) were used to detect the presence of human sVEGFRs in plasma samples.

Statistical Analyses:

Statistical significance was evaluated using Kruskall-Wallis test, followed by Mann-Whitney U test with correction for multiple comparisons. Kaplan-Meier plots and log rank test were used for the analysis of survival. Results are expressed as mean 6 SEM. A value of p<0.05 was considered as statistically significant.

Results

Transgene Expression

Plasma sVEGFR-1, sVEGR-2 and sVEGFR-3 levels were detectable at all time points by enzyme-linked immunosorbent assay (FIG. 31 in Supporting Information). The levels were highest at day 6 after the gene therapy. Plasma level of sVEGFR-1 was minimum of 30.5 ng/ml at day 27 after the treatment, whereas plasma level of sVEGFR-2 was over 3106.7 ng/ml throughout the follow up. Plasma level of sVEGFR-3 was 18.1 ng/ml at the time of sacrifice. In the control group, no signals were detected for the soluble receptors at any time point. The plasma levels of sVEGFR-1, -2 and -3 behaved the same way as in our earlier study with soluble receptors.¹² Reverse-transcription PCR with 35 cycles has confirmed mRNA expression of all trangenes in liver samples 6 days after the gene transfer (data not shown).

Intraperitoneal Tumor Growth

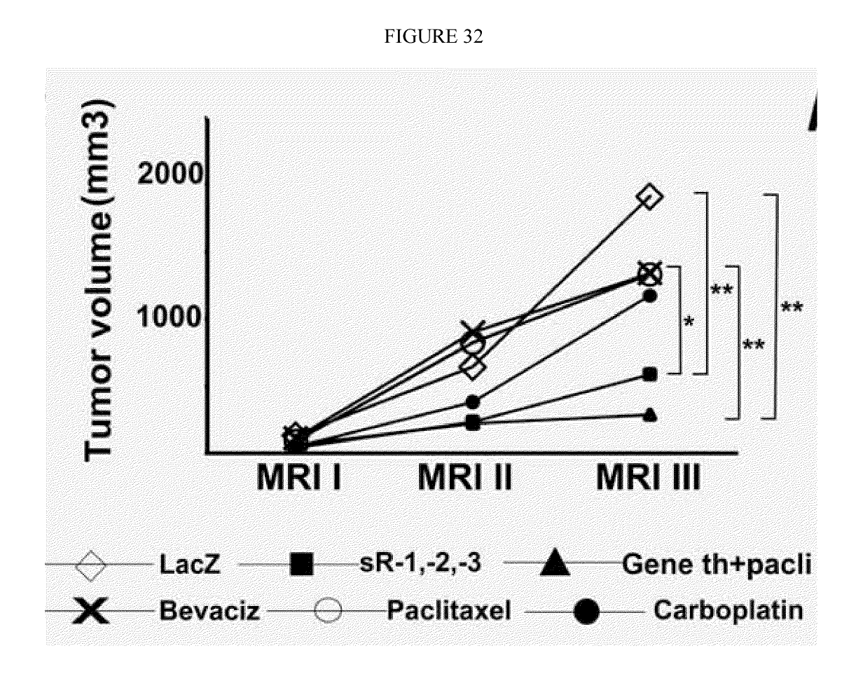

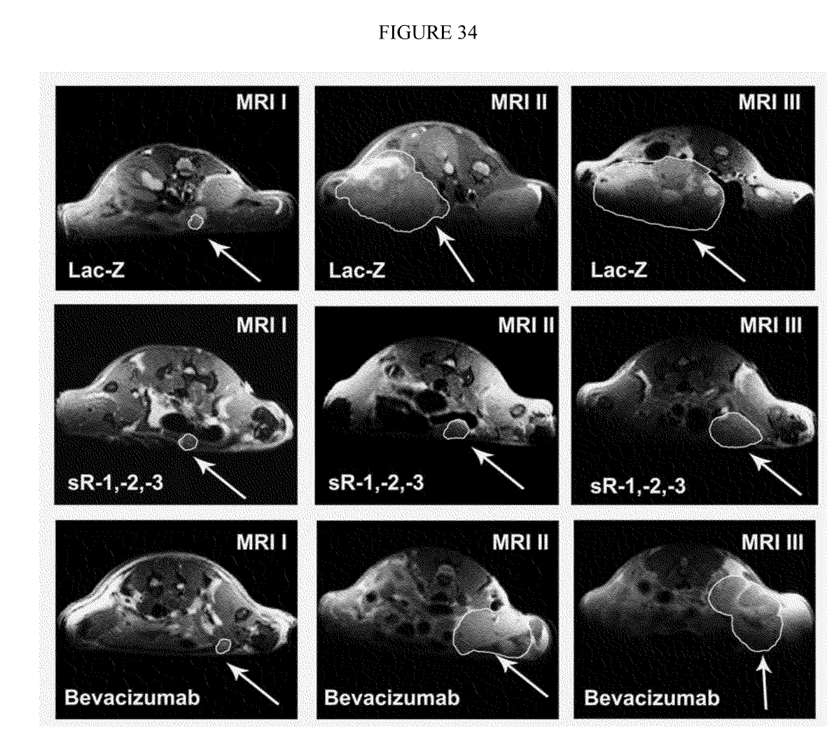

All mice developed intraperitoneal tumors within 3 weeks (6-21 days) after SKOV-3m cell inoculation. Sixty-two percent of the mice were treated within 10 days and 35% in 11-14 days after the inoculation of the SKOV-3m cells. At the baseline, tumor volumes detected by MRI did not differ among the controls and different treatment groups. MRI was repeated weekly after the treatment. In the second MRI (after 1 week of the treatment), the mean tumor volumes of mice treated by gene therapy and paclitaxel were significantly smaller compared to control mice or mice treated with bevacizumab or paclitaxel (p=0.001). Tumor volumes in the gene therapy group were also significantly smaller than in the control (p=0.014), bevacizumab (p=0.0005) and paclitaxel groups (p=0.006; FIGS. 32 and 34).

At the time of the third MRI (2 weeks after the treatment), the mean tumor volume in the combined gene therapy and paclitaxel group was 85% smaller (p=0.002) than in the control, 79% smaller than in bevacizumab (p=0.006) and paclitaxel (p=0.006) groups and 76% smaller than in carboplatin group (p=0.046). At the same time point, tumor volumes were also significantly smaller in the gene therapy group compared to the control, bevacizumab and paclitaxel groups (p=0.004; p=0.036; p=0.036, respectively; FIGS. 32 and 34).

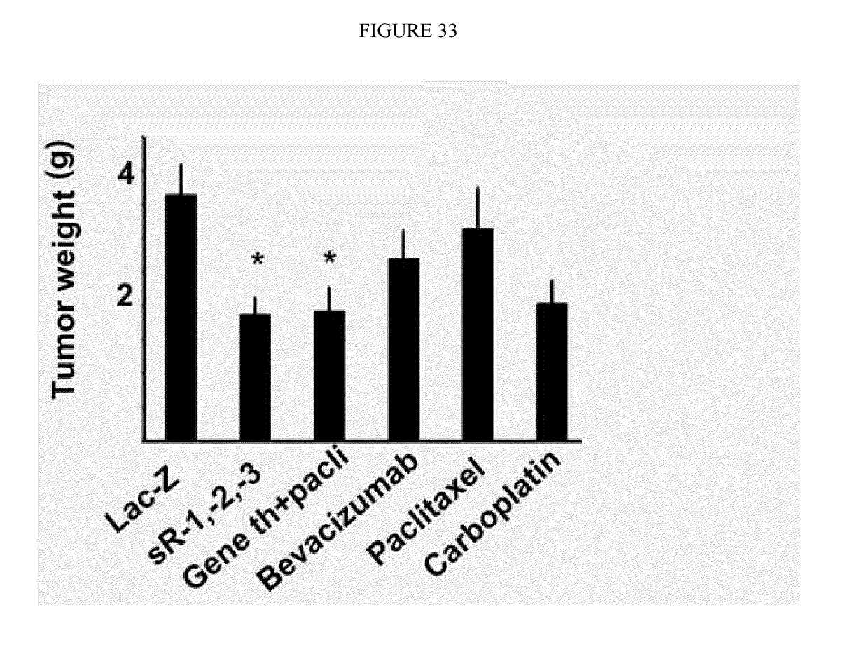

At the end of the follow-up, the final mean tumor weights of the mice treated by gene therapy were 50% smaller (p=0.021) than in the controls (1.8 g vs. 3.6 g). Additionally, tumors of the gene therapy group were 42% smaller than in the paclitaxel group (p=0.02). Mice treated by gene therapy and paclitaxel had significantly smaller tumors than the controls and the paclitaxel alone (p=0.037, p=0.048, respectively; FIG. 30, FIG. 33).

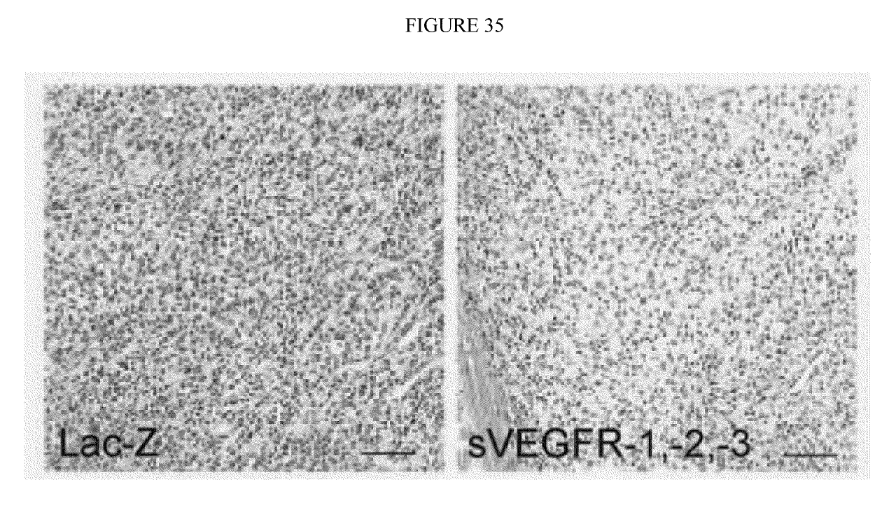

In this animal model of ovarian cancer, tumors were poorly differentiated (grade 3) serous cystadenocarcinomas with variable size of nucleus and limited stroma. In mice treated by gene therapy, tumor tissue was partly replaced by connective tissue, and morphology of the tumors was disturbed (FIG. 35). Focal necrosis and connective tissue were present in the tumor tissue in gene therapy group (sVEGFR-1, -2 and -3). The cell proliferation index measured by Ki-67 staining did not show any difference according to the treatment arm nor was there any significant difference in the amount of ascites formation. Lymphatic vessel density measured by the LYVE-staining did not differ significantly between the treatment groups, although it tended to be less intense in the gene therapy group (data not shown). In control tumors, both human and mouse VEGF-A and PLGF were the most expressed VEGF types. VEGF-D expression was not detectable in cells or tumors (data not shown).

Microvessel Measurements

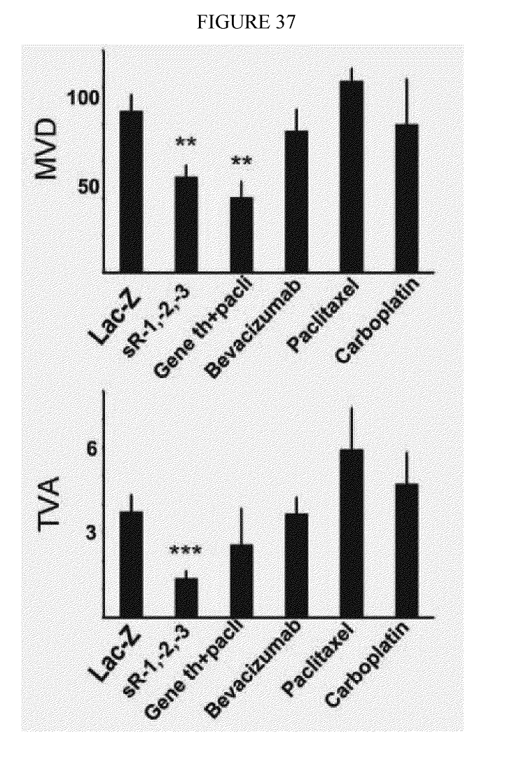

To detect the effect of the treatment on intratumoral micro-vessels, MVD and TVA were measured. Both MVD (51.59%±6.15%) and TVA (1.37%±0.25%) of the tumors in the gene therapy group were significantly smaller than in the control (87.04%±8.66%; p=0.001 and 3.74%±0.57%; p=0.0005) and paclitaxel groups. Compared to controls and the paclitaxel group, significantly lower MVD of the tumors in the gene therapy combined with chemotherapy was also observed (p=0.008; p=0.02, respectively). However, bevacizumab did not have any effect on angiogenesis by micro-vessel measurements (FIGS. 36 and 37). TVA of the tumors in the gene therapy group was also significantly smaller than in the bevacizumab and carboplatin groups (p=0.005; p=0.002, respectively; FIG. 37).

Survival and Safety

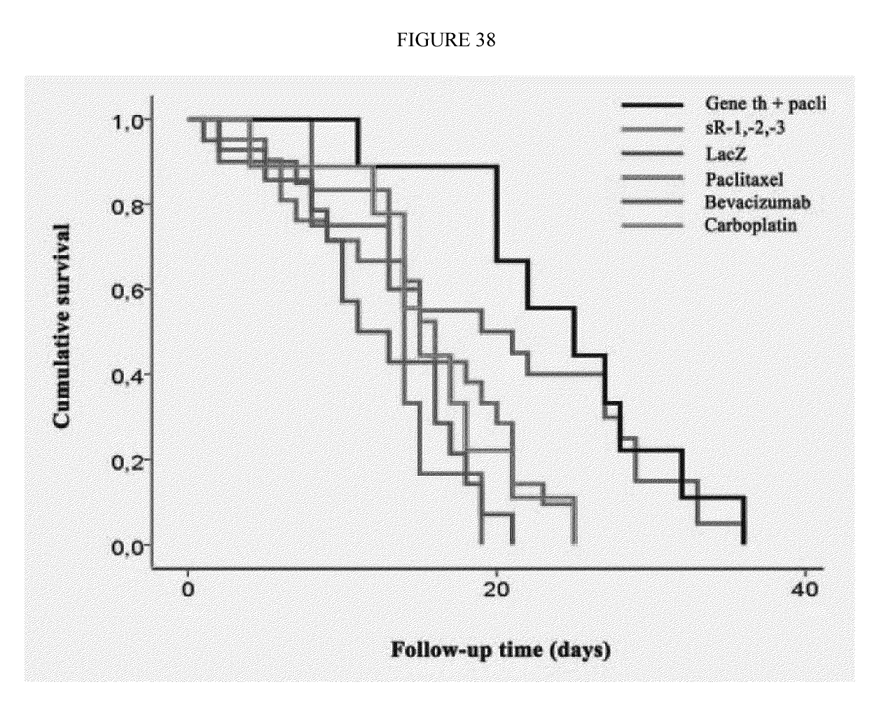

The mean survival (days) was significantly longer in the combined gene and chemotherapy group (25±2 days) compared to the control group (15±1 days) and the other treatment groups (p=0.001). The mean survival of the gene therapy group (19±2 days) was also significantly prolonged compared to the bevacizumab group (p=0.05). On the contrary, the mean survival of the bevacizumab group (12±1 days) did not differ significantly from the survival of the control group. Overall, gene therapy with paclitaxel prolonged the survival of mice compared to all other treatment groups (p=0.001). The mean survival times of the other treatment groups were as follows: 15±1 days in paclitaxel and 13±1 days in carboplatin group (FIG. 38). Overall, prolongation of the survival was also observed by gene therapy compared to bevacizumab (p=0.05).

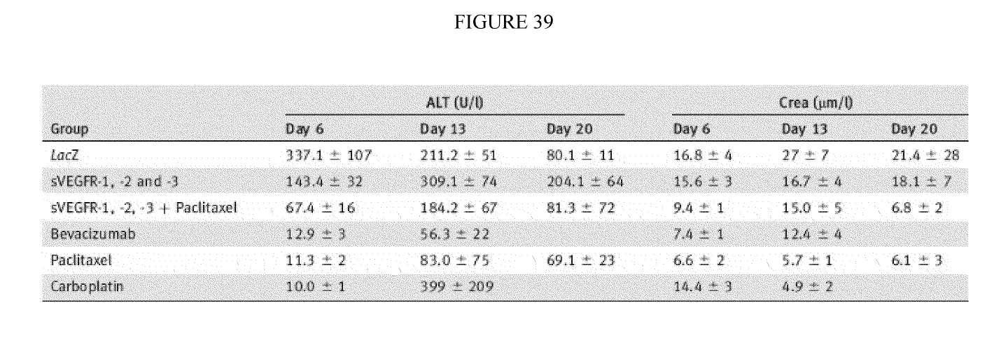

Safety was judged by the assessment of the histological samples of liver, spleen, kidneys and lungs as well as by the analysis of plasma ALT and Crea levels. Liver samples of both treated and control mice were normal 6 days after the gene transfer. At the end of the follow up, there was evidence of regenerative changes in the control and gene therapy-treated groups, which consisted of stronger atypical changes, large variation in shape and size of the nucleoli and local necrosis especially in the combination gene and chemotherapy group (data not shown). Plasma ALT levels were elevated at the end of the follow-up time in both treatment and control groups. By that time, there were wide metastatic changes in the liver. In most of the treatment groups, the raise of the ALT levels seemed to be transient with the highest level 13 days after the treatment. Overall, ALT rise was associated with gene therapy, and no significant difference in ALT levels could be observed between LacZ, sVEGFR or sVEGFR together with the paclitaxel treatment arms. Creatinine values were within normal range (FIG. 39). Neither was there macroscopic nor microscopic alterations observed in the other organs (data not shown).

Discussion

In this study, we demonstrate a survival advantage of antiangiogenic sVEGFR gene therapy together with paclitaxel chemotherapy. Control arms included chemotherapy regimens, which are included in the standard treatment of epithelial ovarian cancer as well as bevacizumab, the VEGF-inhibiting antibody. In a mouse model of macroscopic ovarian cancer detected by MRI imaging, the mean survival was significantly better in mice treated by gene therapy combined with chemotherapy compared to the control and other treatment groups, the mean survival advantage being 67%. Gene therapy was also more efficient than the anti-VEGF-antibody in the treatment of ovarian cancer.

The antiangiogenic effect of VEGF receptors 1, 2 and 3 is due to a decoy effect of the soluble receptors. VEGFR-1, -2 and -3 have high-binding activity toward VEGFs, but they have no signal transduction domains. They act as VEGF antagonists by competing with the native VEGF receptors and inhibit angiogenesis and new vessel formation, which is vital for the tumor growth and metastasis. This VEGF inhibitory effect was observed by the lower MVD as well as smaller vascular area of the tumors. Additionally, nutritional depletion was noticed as a diminished tumor volume and weight by sVEGFR gene therapy.

The standard first-line cytotoxic chemotherapy of ovarian cancer consists of paclitaxel and platin-based compounds, mainly carboplatin. Studies have concluded that both cisplatin and paclitaxel arrest the cell cycle at G1 or G2/M followed by double-stranded DNA brakes consistent with apoptosis. The additional effect of VEGF-targeted therapy on the cytotoxic chemotherapy has been hypothesized to be a result of vessel normalization after VEGF inhibition although multiple other mechanisms may exist. In this animal model, we could demonstrate a prolonging effect on survival by adding chemotherapy to sVEGFR treatment. The mean survival was 32% longer in the gene therapy combined with chemotherapy arm than in the gene therapy alone. Our results suggest that gene therapy can be added to the chemotherapy without any major toxicity also in ovarian cancer.

The monoclonal anti-VEGF-antibody, bevacizumab, dosed two times per week, did not have any significant effect on survival, tumor volume or mean tumor weight. Bevacizumab is most effective against human VEGF, but it reacts less well against mouse VEGF. However, because SKOV-3m cell line is from human origin, bevacizumab should neutralize VEGF produced by these cells. Mouse-derived VEGF-A secreted from bone-marrow derived cells, fibroblasts and other cells in tumor microenvironment contributes to the progression of carcinogenesis. Therefore, it could be possible that the efficacy of this antibody was underestimated in this study. It may be possible that dosing by intraperitoneal route in this aggressive model with fast growing tumors is less effective than systemic VEGF targeting. Another reason for poor treatment effect may be that in our study, the mice had macroscopic tumors at the time of treatment compared to microscopic phase of the tumor progression in earlier studies. In addition, we did not observe any significant difference in ascites formation. However, the dose as well as dosing schedule of bevacizumab was similar in this work compared to others, where positive effects have been observed.

In human studies, bevacizumab as a single agent has shown comparable activity to chemotherapeutic single agents in recurrent ovarian cancer. In the clinical phase, bevacizumab is mainly used for consolidation and maintenance treatment as well as treatment for ascites. Soluble VEGF decoy receptor (VEGF Trap) combined with paclitaxel has prolonged the survival and has shown its antiangiogenic influence on tumor growth and ascites formation. In that study, the mice were treated intraperitoneally 2 weeks after the inoculation of the tumor cells without confirming whether there was a visible tumor or not. Combination treatment had also an influence on tumor metastasis and induction of cell apoptosis. Other studies with soluble VEGFR-1/Flt-1 have also shown efficacy on tumor growth and ascites formation. Tumor cells were injected both subcutaneously and intraperitoneally, and there was no survival benefit in the i.p. group. Several small molecule tyrosine kinase inhibitors targeting VEGFRs have been investigated in phase II studies in relapsed ovarian cancer with some response, but the duration of the effect has been limited, and the continuous dosing of the regimen is being explored.

Liver toxicity has been reported previously when adenoviral sVEGFR-1 has been used intravenously. According to our earlier study, the histology of the liver samples was normal at the time of the highest sVEGFR-1 levels. However, there were regenerative changes in the liver samples of the mice treated by gene therapy at the end of the follow-up. These findings may also be contributed by severe, widely metastasized intraperitoneal carcinosis. In this study, we used the maximum doses of adenoviral sVEGFRs, although lower levels of soluble receptors might reduce liver toxicity without compromising the treatment effect. Liver enzyme levels may also reflect the very aggressive behavior of the SKOV-3m cell line, the first visible tumors arising usually <10 days after inoculation of the cells. The highest ALT levels were measured 13 days after the treatment and before sacrifice in all study groups (FIG. 39).

This cancer xenograft model resembles the clinical setting of human ovarian cancer with wide intra-abdominal metastasis. The diagnostic and regular monitoring of the tumors in mice by MRI and gene therapy dosed intravenously makes this model also more challenging to establish. In conclusion, we show a survival benefit of up to 67% after antiangiogenic gene therapy with the combination of sVEGFR-1, -2 and -3 and paclitaxel compared to the controls, those receiving the antiangiogenic treatment with the monoclonal antiVEGF-antibody bevacizumab or single chemotherapy.

Ovarian, Breast and Pleural Cancers

We first tried to treat malignant cancers on the pleura with a viral vector containing a thimidine kinase gene (a “suicide gene”) as the transgene. We found such treatment somewhat effective in producing tumor regression. Surprisingly, however, we found that the transgene had in fact only a limited amount of expression, but patients expressed higher anti-tumor antibodies. We thus felt the positive clinical responses might not be caused directly by tumor eradication by the thymidine kinase suicide gene, but perhaps by an enhanced immunological response induced by the adenoviral Ad.HSV-tk gene therapy vector.

To test this theory, we first re-examined intra-tumoral treatment using viral gene therapy vectors. Gene therapy is used to transfect with an agent which sensitizes the patient against the tumor. Ideally, an anti-cancer gene therapy vector would do two things. First, it would release a variety of tumor cell antigens, allowing the patient's host immune system to “select” or identify appropriate antigenic targets. Second, it would allow an antigen to be presented in an “immunogenic” fashion: that is, it would provide the host immune system with strong danger signals, and would overcome tumor-induced immunosuppression, and would activate a variety of immune cells, especially dendritic cells and T lymphocytes. To achieve this, we posited that one would need both a correct type of vector and a correct type of transgene. For vector type, we posited that, in contrast to the prior art, which teaches that a host anti-vector immune response dampens or destroys viral gene therapy effectiveness, we would want to test an immunogenic vector. In addition, the vector would need to have high in vivo expression. Both of these qualities implied that such a vector would strongly induce danger signals. We posited that among known viral vector types, adenovirus was both highly immunogenic and enjoyed high in vivo expression, and adeno-associated virus and lentivirus may be less so.

Regarding the correct transgene, we posited that an optimal transgene would have broad immunogenicity and host-immune response activation activity. We posited that an interferon transgene seemed potentially suitable, as interferon might have several different, mutually-reinforcing activities: inducing an indirect anti-tumor response by activating natural killer cells, macrophages and cytotoxic T lymphocytes; inducing potential anti-angiogenic activity which, by decreasing blood supply to a solid tumor (which we explored as effective alone to a certain extent), would inhibit solid tumor growth; and direct killing of human tumor cells, primarily via apoptotic cell death.

Two apparently suitable candidates were recombinant adenovirus carrying an interferon beta transgene (Ad.IFN-β) and recombinant adenovirus carrying an interferon alpha-1 transgene (Ad.IFN-α11). These products each have a high viral titer. They each are reported to efficiently transduce both growing and post-mitotic cells. Their viral and transgene DNA does not integrate into the host cell nucleus, assuring transient (rather than permanent) expression. Further, they each are immuno-stimulatory (producing a material anti-viral host immune response) and cytotoxic.

We have to-date performed three Phase I human clinical trials on a total of twenty-seven patients with malignant forms of organ cancer, and in twenty-five patients identified as having an accessible mesothelium-lined pleural space, using Ad.IFN-beta.

The 27 total patients included 17 cases of malignant mesothelioma, 5 cases of lung cancer, 3 cases of ovarian cancer and 2 cases of breast cancer. These cancers, while of diverse location (with the exception of breast), share the common feature of being in organs located in body cavity lined with mesothelium.

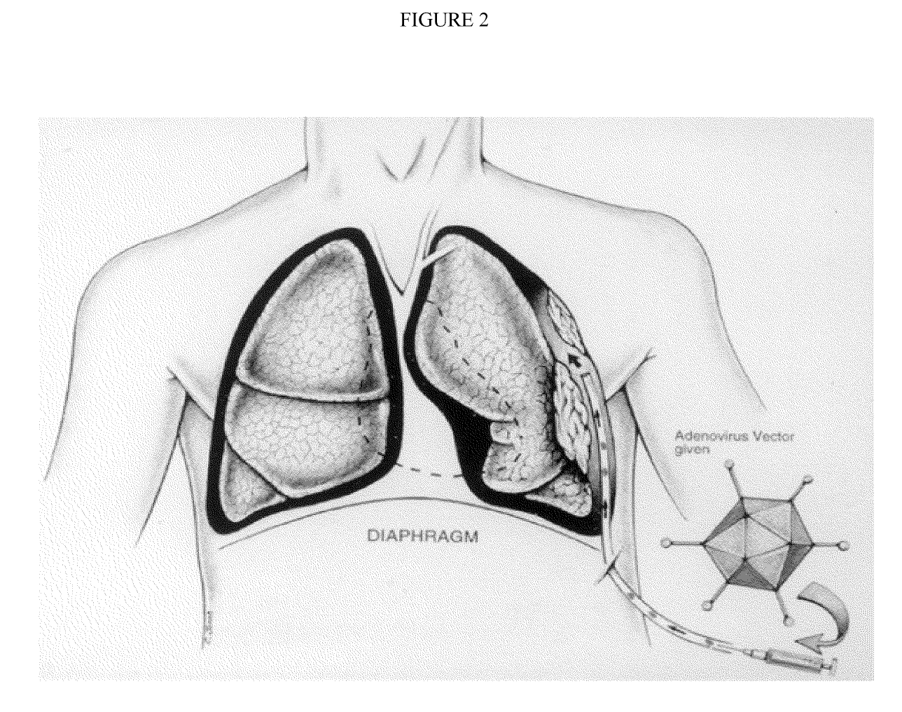

Using a PLEUR-X® intrapleural catheter (commercially available from CareFusion Corporation, Waukegan, Ill.) we irrigated the pleural space with a solution of the viral vector. See FIG. 2. For patients in the first trial, we administered only one dose of viral vector; for patients in the second and third trials, we followed the first administration with a second, repeat administration spaced 7 or 14 days after the first dose. We used doses ranging from 3×10e11 viral particles to 3×10e12 viral particles twice. We then monitored the patients' clinical course, including assaying their pleural fluid for gene expression. Our primary purposes in doing this study were to (1) evaluate whether viral vector remains safe and non-toxic when administered into the pleural cavity, (2) determine an appropriate dose range, and (3) measure gene transfer from vector to host cells. Our secondary goals were to (4) assess any cytokine and/or immune responses to the vector or the interferon-beta expressed by the transgene, and (5) measure tumor responses (if any) to this intervention.

We found that viral vector administered to these administration sites is overall very well tolerated; most patents developed transient lymphopenia and fever with mild hypoxemia. Our dosing did not reach a maximal tolerated dose. Measuring interferon-beta levels in pleural fluid clearly showed successful gene transfer and expression from the first administered dose. Most mesothelioma patients also showed clear antibody responses to mesothelioma antigens. In contrast, the second dose of adenoviral vector (14 days after and 7 days after the first dose, in the second and third clinical trials, respectively) appeared completely ineffective in producing interferon-beta; the amount of interferon-beta detected did not measurably increase in response to the second dose at either timing. We believe this is due to a rapid (within 7 days) induction of a host anti-adenovirus immune response which produces adenovirus-neutralizing antibodies in the pleural fluid.

Almost all patients also showed a humoral anti-tumor immune response. We measured this by diluting patient serum samples (both pre- and post-treatment) and using the diluted sera to PAGE immunoblot both purified mesothelioma antigens (the SV40 and the 40 kD mesothelin antigens) and tumor cell lines. The immnoblots showed binding activity post-immunization at a number of sizes indicative of mesothelioma and tumor antigens. For example, we found pronounced binding post-immunization at a band at the location one would expect to find the 40 kD mesothelin antigen. We thus posit that the immunization provokes host production a specific antibody response not only against the adenoviral vector, but against cancer antigens as well.

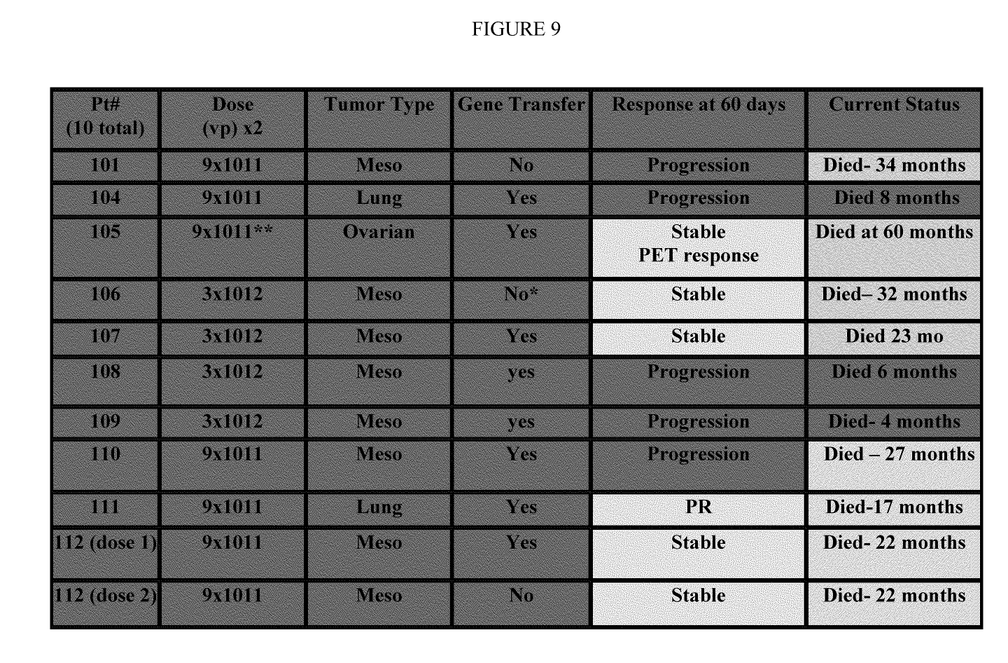

Our data for the first trial (using one dose of viral gene therapy vector) is shown in FIG. 9, Clinical Data: First Trial—One Dose (February 2011).

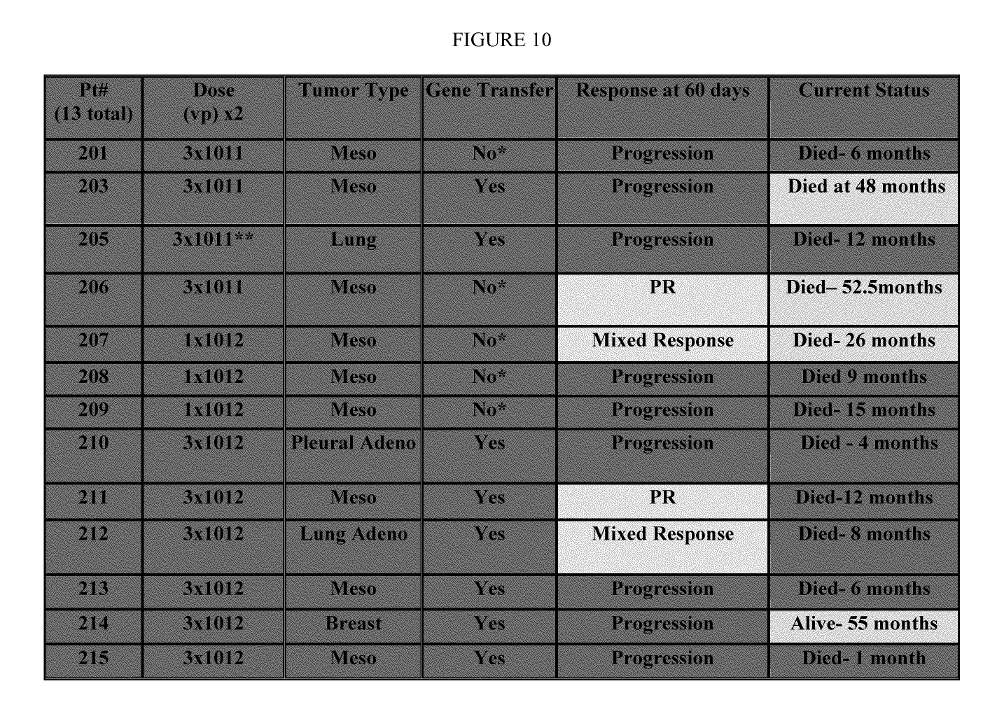

Our data for the second trial (using two doses of viral gene therapy vector administered in a two-week interval) is shown in FIG. 10, Clinical Data: Two Doses—Two Week Interval (February 2011).

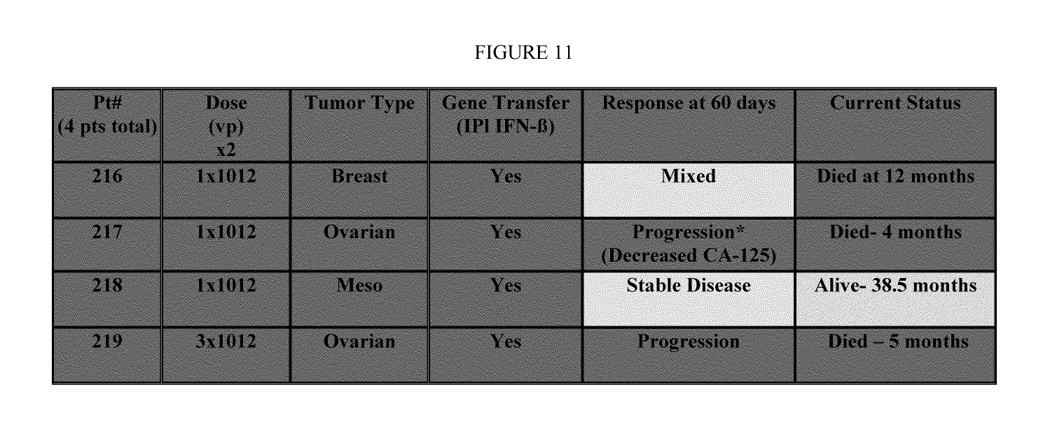

Our data for the second trial (using two doses of viral gene therapy vector administered in a one-week interval) is shown in FIG. 11, Clinical Data: Two Doses/One Week Interval (February 2011).

Our findings demonstrate superiority of adenoviral Ad.IFN gene therapy compared to conventional cytotoxic chemotherapy. Expressed as the median survival time (the time at which 50% of patients remain alive), cisplatin achieves a 9 month median survival time, adding pemetrexed increases this to 12 months, and we found that Ad.IFN gene therapy monotherapy increased this to 22 months.

We repeated this approach with a similar vector, Ad.IFN-alpha-2b, in nine patients with malignant pleural mesothelioma. We gave three of these patients two doses of 1×10e12 viral particles. We separated these two doses by only three days (compared with the seven and 14 day separations we previously used). These patients showed extremely high levels of interferon-alpha, with a “bump” or sharp increase after the second dose. These high levels of interferon were associated with interferon-like syndrome: fatigue and muscle aches.

For the subsequent patients, we thus decreased the dose by nearly an order of magnitude, to 3×10e11 viral particles. Every patient showed, on a PAGE-immunoblot assay, increase in antibodies specific for the 33 kD osteopontin antigen. Thus, even with this decreased viral gene therapy vector dose, we found that every patient generated antibodies against the osteopontin antigen, a marker we believe, for the instant purposes, indicative of mesothelioma cell lines.

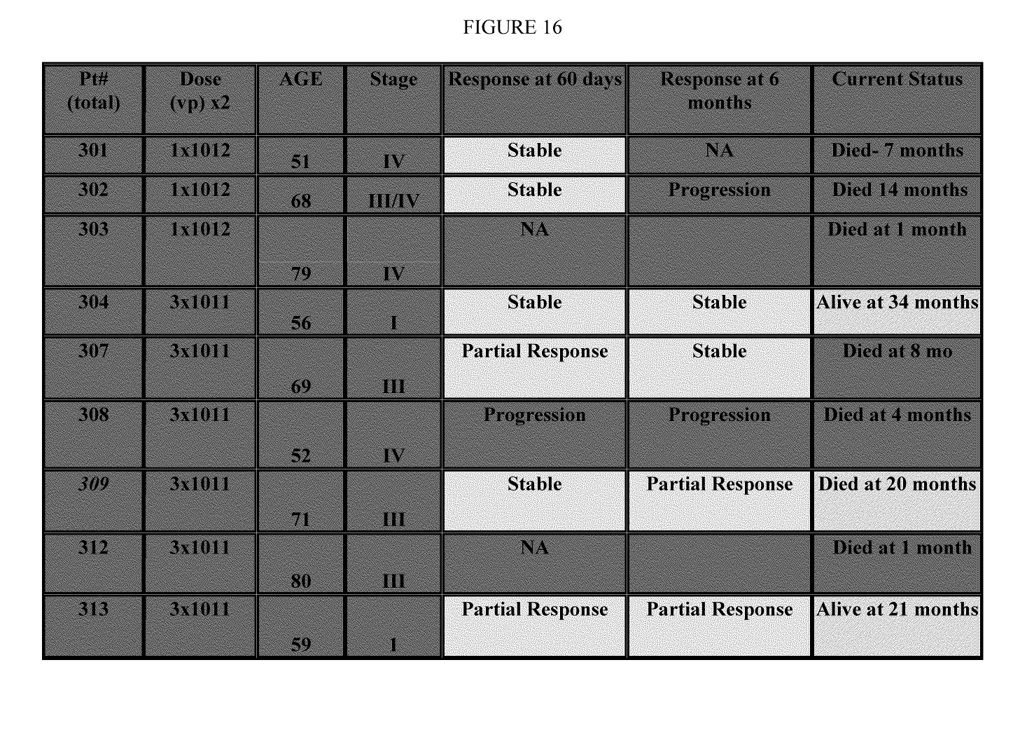

Similarly, FIG. 16 shows activation of NK cells by interferon gene therapy vector in Patient #309. We measured peripheral blood mononuclear cells from a pre-treatment blood sample, and from a blood sample taken two days after gene vector administration, using flow cytometry. We identified NK cells on the basis of the cell surface expressions of CD56 and CD16 after gating on the CD3−/CD14−/CD19−/CD20− lymphocytes. Shown in the Figure are CD3−/CD14−/CD19−/CD20−/CD56^{d/m}/CD16+ cells expressing the activitaion marker CD69 and IFNαR, before gene transfer (left chart) and 2 days after gene transfer (right chart). Numbers in the smaller font in the corner of each quadrant represent % of each subset in the parent gate, while numbers in the larger font in the middle of the upper right quadrant represent % of activated NK cells (CD3−/CD14−/CD19−/CD20−/CD56^{d/m}/CD16+/CD69+/IFNαR+) in the lymphocyte gate. Note the marked up-regulation of the activatin marker CD69 in the post-treatment sample.

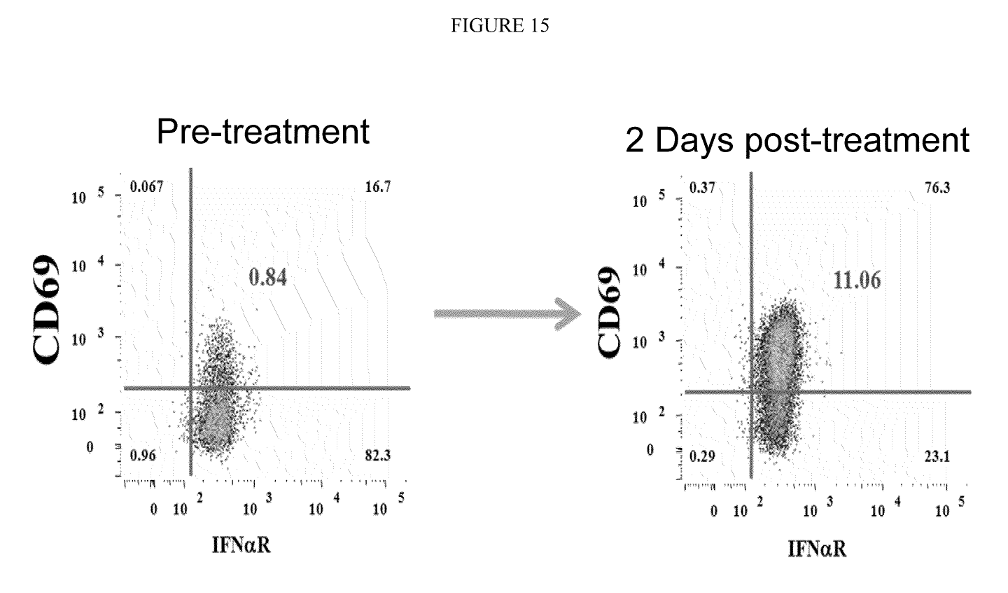

We found that adenovirally-administered interferon gene therapy induces systemic NK cell activation. Using CD16+/CD56+ PBMCs, the ratio of CD69 increased from 0.84 pre-vaccination to 11.06 post-vaccination. See FIG. 15. Our resulting overall results are shown in Clinical Data: Ad.IFNα—two doses: 5-2012. See FIG. 16.

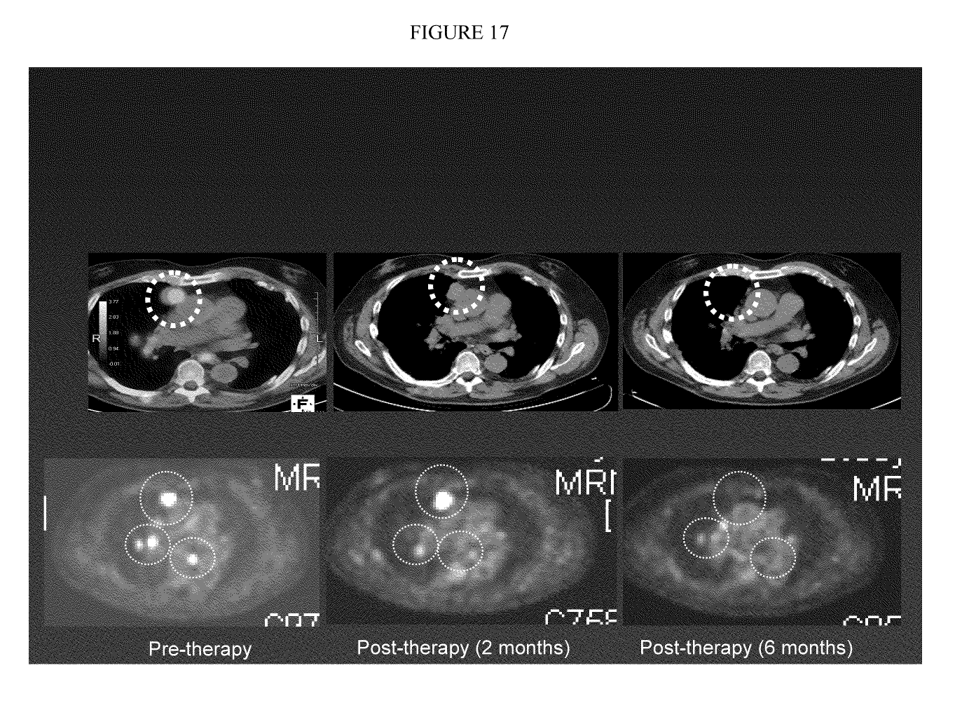

The superiority of gene therapy to prior art cytotoxic chemotherapy is shown, for example, in e.g. FIG. 17. For example, Patient #309 showed major tumor regression 6 months after initial gene therapy with no additional therapy. See FIG. 17.

Despite this success, adenoviral interferon therapy has several limitations. For example, we found that it works well in small tumors, but its efficacy is markedly diminished with larger tumors. Further, viral vectors are thought to be able to be given only once, due to the patient's generation of neutralizing anti-viral antibodies.

While both interferon gene therapy and cytotoxic chemotherapeutic drugs are thought potentially useful for cancer, the art teaches that combining the two would have several disadvantages, notably induction of leucopenia, which would eliminate anti-tumor directed lymphocytes. Our results with mono-therapy, however, hinted that we might be able to maximize therapeutic benefit by using gene therapy in a qualitatively different way. While the prior art teaches its potential use as a cancer cure per se, we proposed using interferon gene therapy to “prime” the patient's immune system into mounting an anti-tumor CD8 immune response, and then maintaining this anti-tumor effect during subsequent courses of chemotherapy. Given the aforementioned shortcomings of gene therapy monotherapy, however, we decided to test the two in combination. We posited this combination could release tumor antigen for presentation to the host immune system (thus inducing a host anti-tumor response), could inhibit the patient's immunosuppressive cells, and perhaps favorably alter the tumor microenvironment.

We thus tested the combination of inter-tumoral administration of interferon-alpha gene therapy combined with chemotherapy. To do so, we injected: (a) AB12 mesothelioma line cells into Balb/C mice; (b) TC1 non-small cell lung cancer cell into C57BL/6 mice; (c) LLC non-small cell lung cancer cell into C57BI/6 mice; (d) Ad.Cre into Lewis lung carcinoma KrasG12D+ mice (to activate the animal's oncogenic, “floxed” mutated Kras gene); (e) TC1 non-small cell lung cancer cell into C57BI/6 mice; and (f) TC1 non-small cell lung cancer cell into C57BI/6 mice.

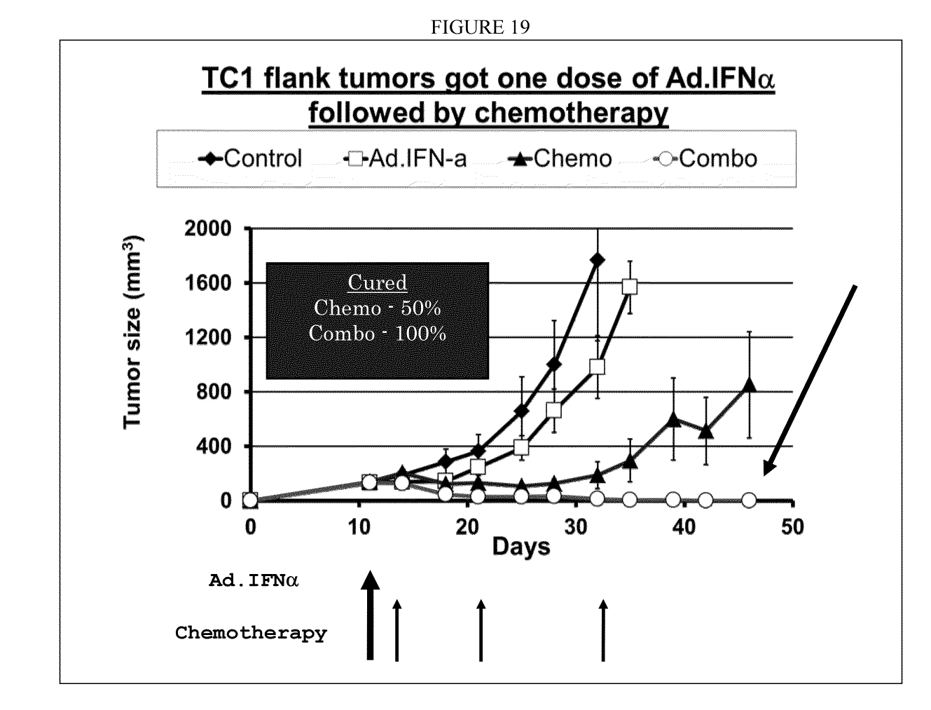

Our preliminary results are shown in FIG. 19. These results are quite surprising on several levels. Our tests showed that one dose of interferon-alpha gene therapy produced outcomes superior to that observed with placebo. Surprisingly, multiple-dose chemotherapy produced superior outcomes than achieved with gene therapy; this result in mice is surprising because it conflicts with our earlier findings, discussed above, which showed that interferon gene therapy in humans is superior to chemotherapy. More surprising, the efficacy of multiple-dose chemotherapy doubles when preceded by a single intra-tumoral dose of interferon gene therapy: chemotherapy alone cured 50% of the mice in our study, while chemotherapy with gene therapy cured 100%.

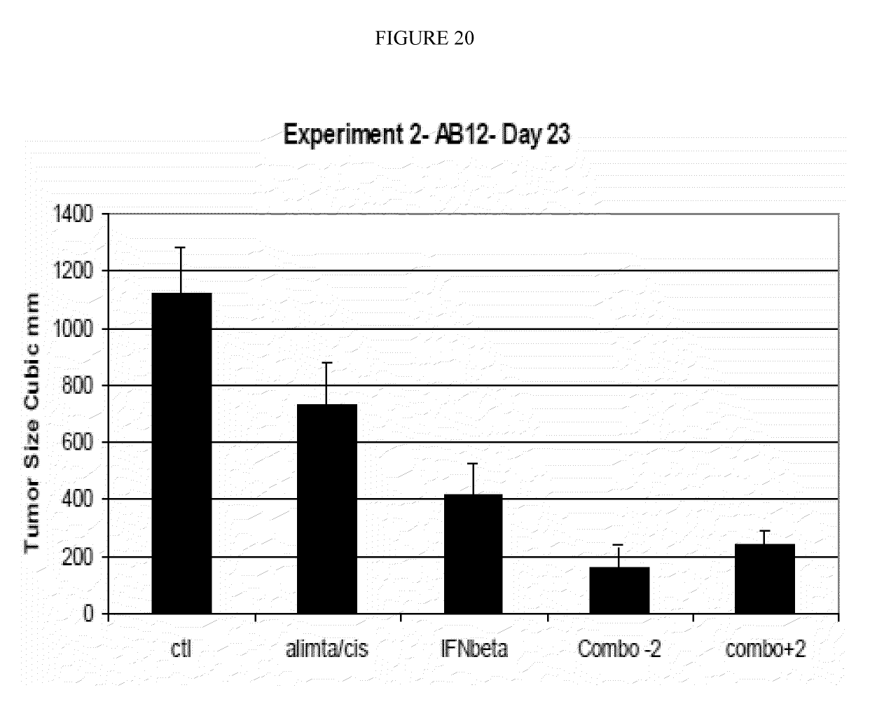

We found that gene therapy augments the efficacy of cisplatin combined with either pemetrexed or cisplatin. Measured 23 days after treatment, average tumor size with placebo treatment was about 1100mm³. Treatment with cisplatin and pemetrexed reduced average tumor size to about 720 mm³; treatment with interferon-beta gene therapy reduced average tumor size to about 400 mm³; combination treatment reduced average tumor size to about 200 mm³. See FIG. 20.

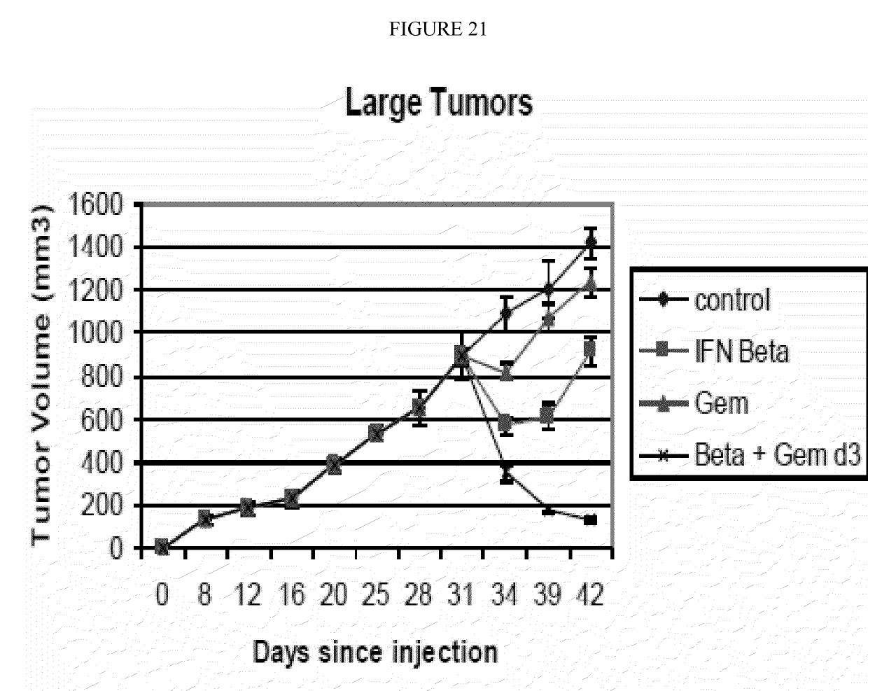

Similarly, using gemcitabine as the chemotherapeutic, we found that after 40 days, average tumor size was about 1400 mm³ with placebo treatment, 1200 mm³ with gemcitabine treatment, 900 mm³ with interferon-beta gene therapy treatment, and about 110 mm³ with combination therapy (interferon-beta gene therapy followed on day 3 with gemcitabine). See FIG. 21. Thus, we found that combining intra-tumoral interferon gene therapy with platinum-compound, gemcitabine and pemetrexed chemotherapies is synergistically effective in treating tumors. Without intending to limit the scope of our appended legal claims, we suspect that this synergy arises from at least four mechanisms. First, we suspect that combination therapy decreases populations of immunosuppressive cells (e.g., myeloid-derived suppressor cells (MDSC), T-regulatory cells and B cells). Second, we suspect that combination therapy causes release of intra-tumor antigen, which in turn stimulates immune memory cells which in turn leads to efficient cross-priming of host immune cells against tumor cell antigens. Third, we suspect that combination therapy alters the tumor micro-environment by increasing production of immune “danger signals” and immunostimulatory cytokines. Fourth, we suspect that combination therapy augments the traffic of T-cells into tumors.

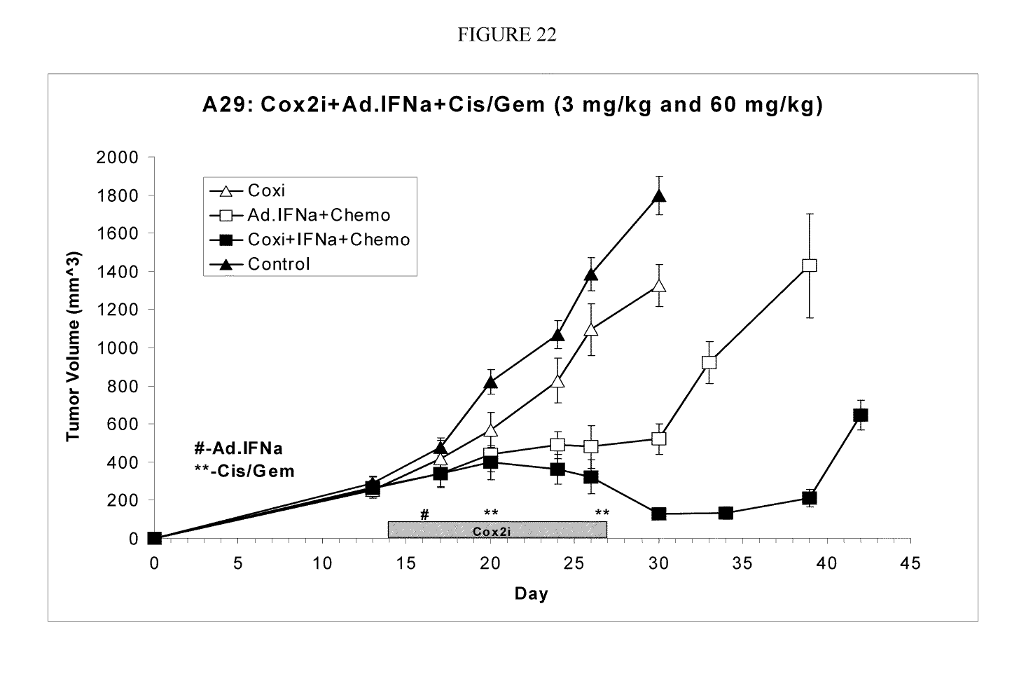

We did a further pre-clinical (animal model) study assessing the combination of interferon gene therapy and chemotherapy and COX-2 inhibitor. Our results are shown in FIG. 22. Injecting a mouse model with tumor cell line at day=0, by day=30 in placebo mice the tumor had grown to about 1,850 mm³. In contrast, administering COX-2 inhibitor from days 14 through 27 somewhat reduced mean tumor size to about 1,300 mm³. Administering intra-tumoral interferon alpha gene therapy (at day 17) followed by a cisplatin/gemcitabine combination (at day 20 and at day 27) dramatically reduced mean tumor size, to 500 mm³. Combining COX-2 with the interferon gene therapy+chemotherapy combination reduced tumor size to less than 200 mm³.

These results clearly indicate that adding COX-2 to the interferon gene therapy+chemotherapy combination makes the three-way combination more effective. These results also intimate that the two-part combination of interferon gene therapy+chemotherapy might, if used in humans at the correct dose sizes and times, be effective to treat organ cancer, provided the rate of administration is effective.

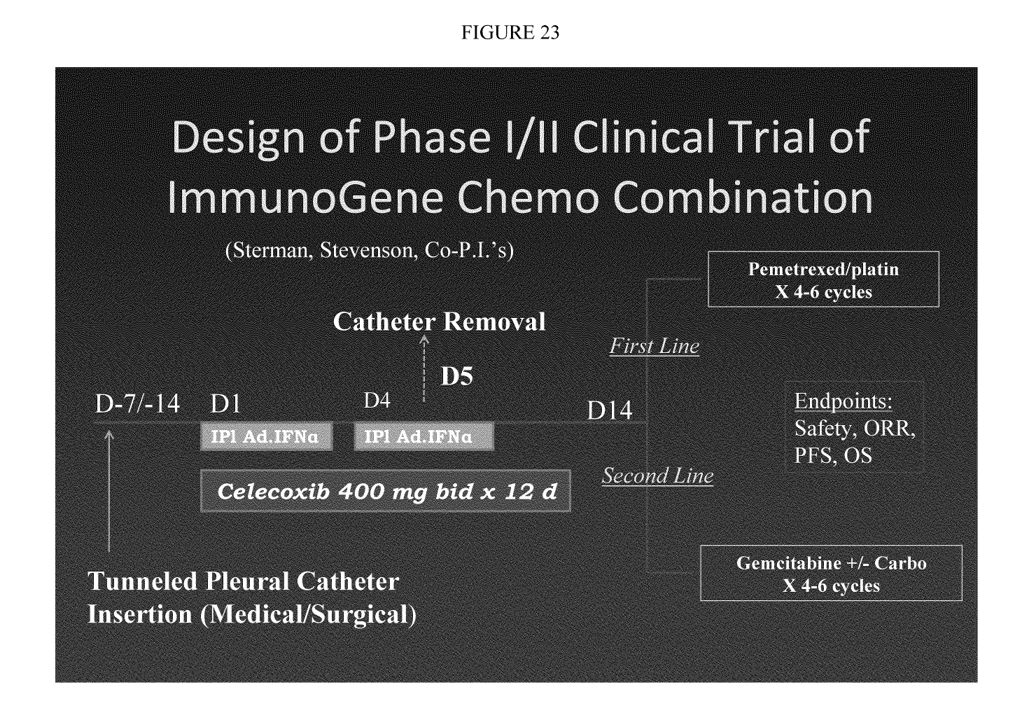

Based on this pre-clinical data showing synergy between interferon gene therapy and chemotherapy, we began a Phase II human clinical trial, administering one dose of adenoviral interferon alpha gene therapy, followed two weeks later by either a cisplatin-pemetrexed combination or a gemcitabine-containing regimen. We illustrate the design of the treatment regimen at FIG. 23. Our treatment regimen has several unusual aspects.

First, our treatment regimen entails washing the mesothelium lining the body cavity housing the cancerous organ with gene therapy vector using a catheter, rather than injecting specifically-identified tumors with a needle or a catheter. This approach contrasts with much of the prior art, which teaches that gene therapy is disease site-specific, so that it must be loaded directly into the tumor or cancerous organ to be treated. Our approach also contrasts with the prior art, which teaches that viral gene therapy vector should be administered inside the solid tumor. Our approach enjoys the advantage of not needing to identify every specific tumor needing treatment, and of recruiting a potentially much larger area of host cells for transformation. Further, our approach, by so widely exposing the patient's mesothelium around the cancerous organ to viral gene therapy vector, induces a strong localized patient immune response, which response augments the efficacy of certain chemotherapeutic compounds to cancerous cells alone.

Second, our protocol entails multiple dosing of the interferon viral vector; once at day=1 and again at day=4. This is then followed at day=14 by the beginning of chemotherapy, given in six cycles. (We do not, however, believe the second dose is required to achieve synergistic efficacy)

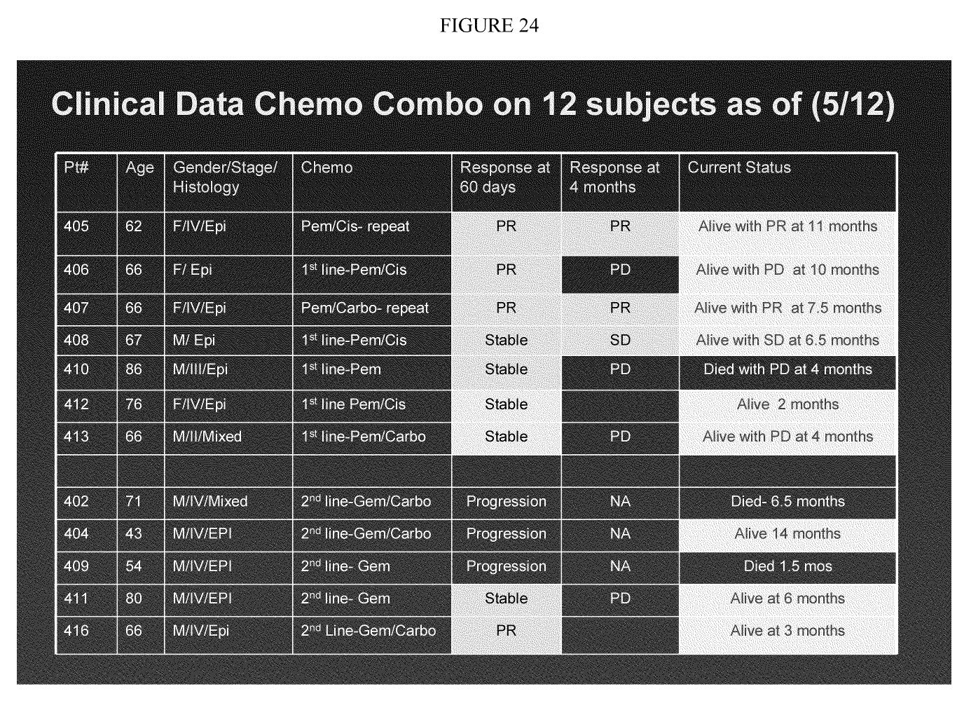

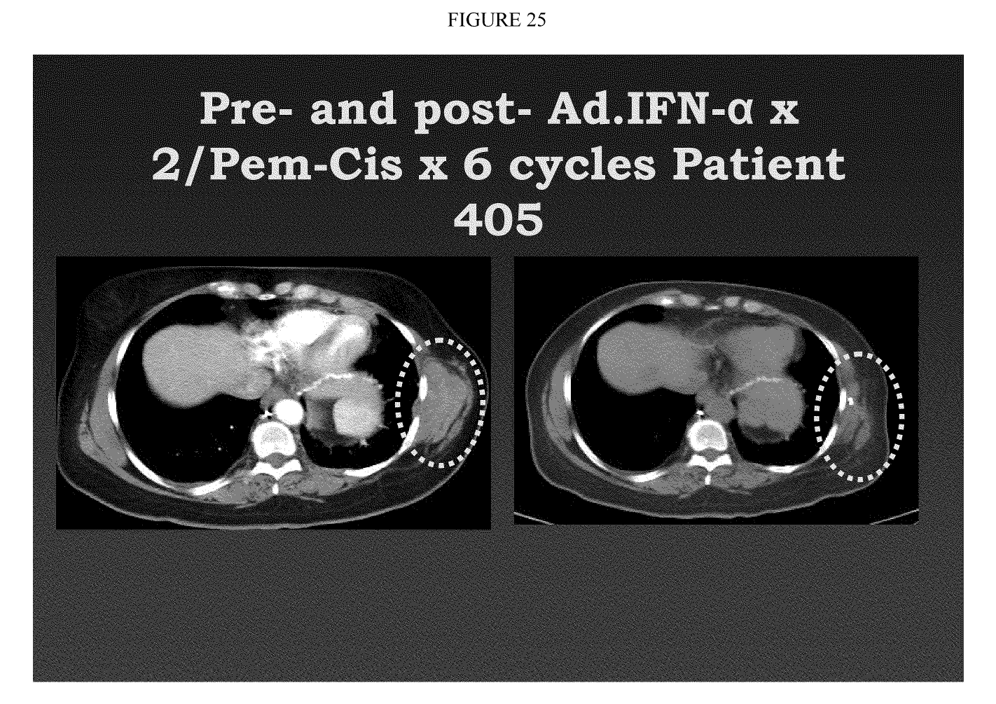

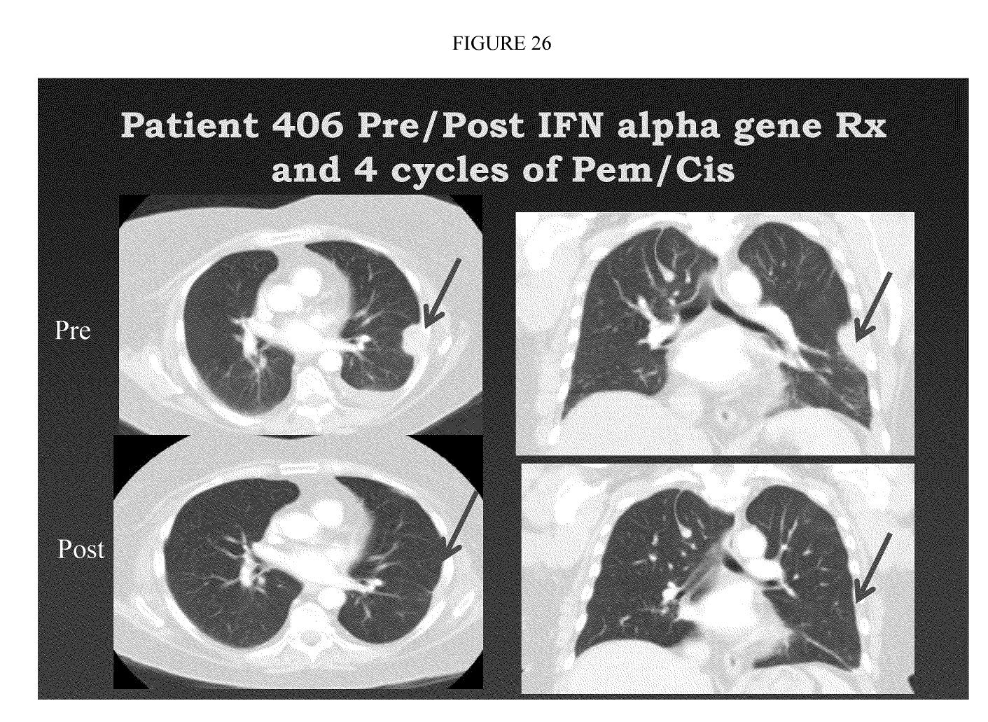

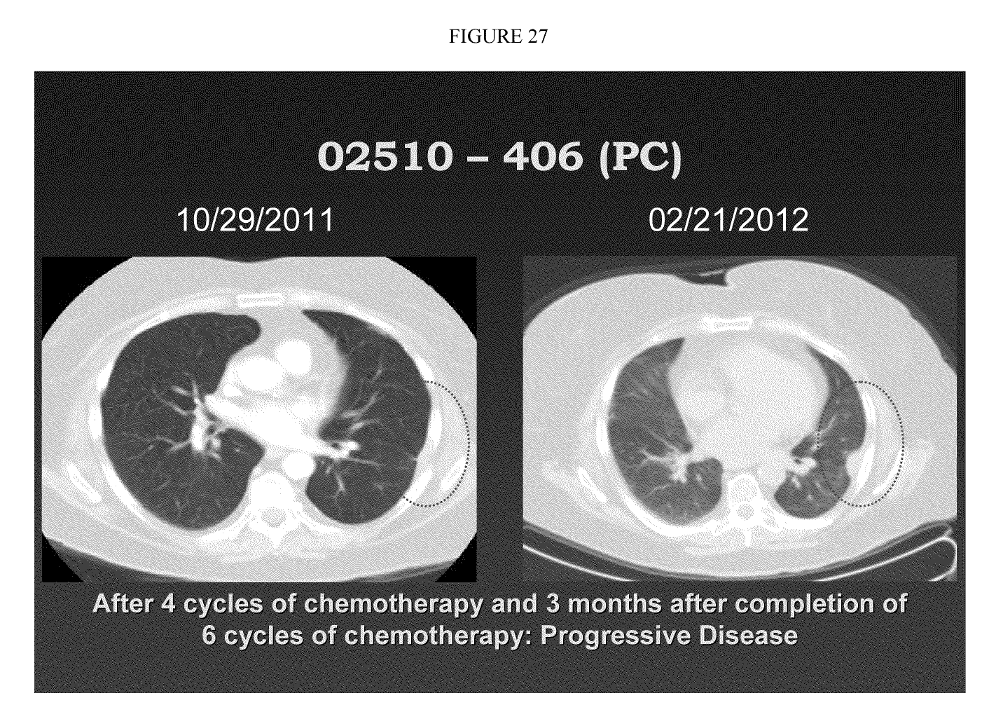

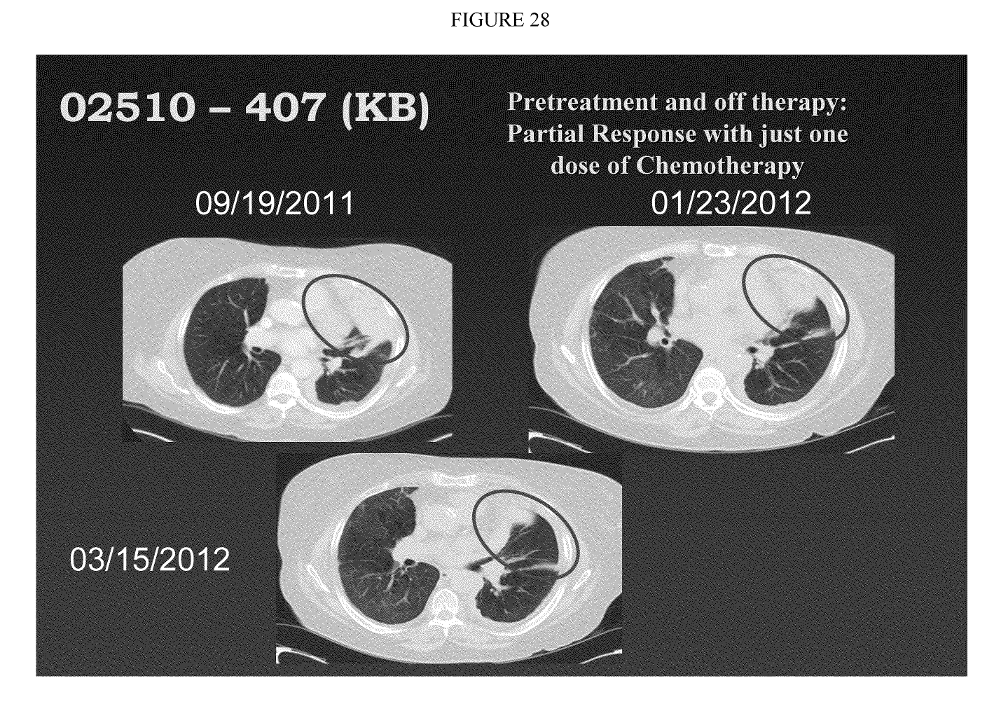

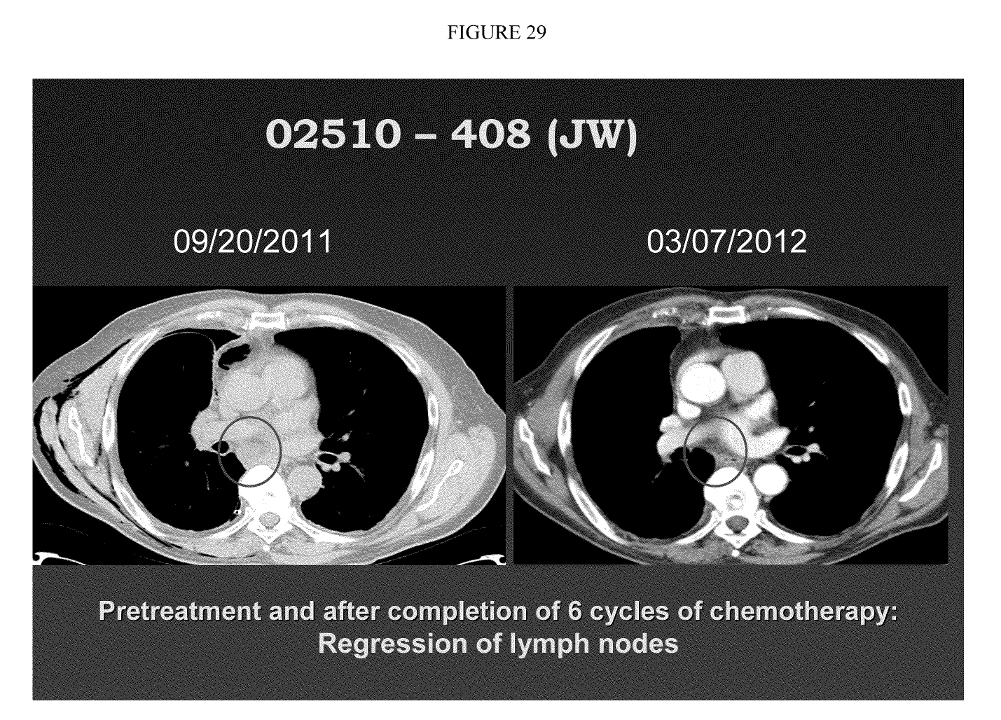

We treated fourteen human patients using this protocol. We found that at 60 days, front line chemotherapy (cisplatin+pemetrexed) produced disease control in 100% of the patients (n=7), with “stable” disease in about half (n=4) and “partial response” in about half (n=3). In contrast, second line chemotherapy (gemcitabine regimens) achieved disease control in only 40% of patients (n=5). We provide results for each patient in FIG. 24. FIGS. 25-29 show more detailed results for several specific patients.

Our results show that intra-tumor administration of gene therapy with an adenovirus expressing Type 1 interferon transgenes can induce an anti-tumor immune response, and clinical responses in some mesothelioma patients. We have also found that combining adenoviral interferon gene therapy with chemotherapy shows promising results in a human Phase 2 clinical trial.

### SUMMARY

Our results show that administering a viral gene therapy vector containing an interferon transgene can dramatically increase the efficacy of cisplatin and pemetrexed and, to a lesser extent, carboplatin and gemcitabine. The skilled artisan would not have expected this synergy. Indeed, the prior art teaches that interferon-based gene therapy can potentially cause interferon-related, flu-like adverse side effects, so the artisan employing interferon-based gene therapy would have wanted to avoid further insulting the patient with chemotherapy and cause the known reaction of leucopenia, reducing any anti-tumor effect.

Our results also show that this increased efficacy does not require injecting the viral interfron vector directly into the tumor, but rather merely requires irrigating the mesothelium lining the body cavity where the cancerous organ is located. Such body cavities include the pleural cavity (the site of mesothelioma and lung cancer), as well as the cavities housing the kidneys, bladder, adrenal and pancreatic glands, prostate and ovaries. This is surprising because the prior art teaches the need to inject gene therapy vector directly into the tumor, or locally into the cancerous organ; the artisan would not have expected topical application (irrigation) to be effective at all, much less synergistically effective.

Given our disclosure, the artisan could with routine experimentation derive variations of and improvements on our work. For example, in our actual human clinical trials, we used an adenovirus-based gene therapy vector; the artisan could, using routine experimentation, substitute a retrovirus- or lentivirus-based vector for a long-term effect.

Similarly, we have used interferon beta and alpha 2b in our actual human testing, but the artisan could likely replicate our results with any of the currently-approved species of interferon (e.g., alpha 2a, beta 1a), or with other Type I interferon species, or analogs thereto, or indeed with Type II or Type III interferon species as well. Thus, we use the claim term “interferon” to refer generally to any interferon, interferon-like compound, or analog thereof.

Similarly, while the examples discussed here in fact employed a transgene coding for an interferon, one can achieve similar ssynergy using a transgene coding for a vascular endothelial growth factor (VEGF). A number of VEGF species are known in the art, as is their employment in viral vector, and the use of the viral vector to inject transgene directly in to a tumor, or alternatively to inject vector into the tumor bed remaining after tumor resection. We believe that our method of irrigating a mesothelium-lined body cavity with this type of vector (rather than injecting it into a tumor), and combing it with chemotherapy, will be synergistically effective compared to administration of either vector or chemotherapy alone.

As used in the claims, we use the term “mesothelium” to encompass both normal (e.g., healthy) and abnormal (e.g., diseased, damaged by cytotoxic agent) mesothelium.

We thus intend the legal scope of our patent to be defined not by the specific examples recited here, but by the legal claims appended here, and any permissible legal equivalents of these claims.

1. In a method of treating a human diagnosed as having cancerous organ by administering chemotherapeutic agent, the improvement comprising administering to said human a recombinant virus, said recombinant virus comprising a homeomimetic transgene.

2. The method of claim 1, wherein said homeomimetic transgene codes for interferon, and wherein said interferon comprises interferon alpha 2b.

3. The method of claim 1, wherein said cancerous organ is located in a mesothelium-lined body cavity.

4. The method of claim 3, wherein said mesothelium comprises mesothelium surrounding lung and wherein said cancer comprises malignant pleural mesothelioma.

5. The method of claim 1, wherein said chemotherapeutic agent comprises an agent selected from the group consisting of: cisplatin, carboplatin, pemetrexed and gemcitabine.

6. The method of claim 5, wherein said chemotherapeutic comprises cisplatin and pemetrexed.

7. The method of claim 1, further comprising administering to said human a COX-2 inhibitor.

8. The method of claim 7, wherein said COX-2 inhibitor comprises celecoxib.

9. The method of claim 3, wherein said cancerous organ is selected from the group consisting of: lung, kidney, adrenal gland, ovary, prostate, pancreas and bladder.

10. The method of claim 3, wherein said mesothelium comprises pericardium.

11. In a method of treating a human diagnosed as having cancerous organ located in a mesothelium-lined body cavity by administering to said human a recombinant virus comprising a homeomimetic transgene, the improvement comprising administering said recombinant virus by irrigating at least part of said mesothelium lining said body cavity of said human with a solution comprising said recombinant virus.

12. The method of claim 11, wherein said cancerous organ is selected from the group consisting of: lung, kidney, adrenal gland, ovary, prostate, pancreas and bladder.

13. The method of claim 12, wherein said mesothelium comprises mesothelium surrounding lung and wherein said cancer comprises malignant pleural mesothelioma.

14. The method of claim 11, wherein said homeomimetic transgene codes for interferon, and wherein said interferon comprises interferon alpha 2b.

15. The method of claim 11, further comprising administering to said human a chemotherapeutic agent.

16. The method of claim 15, wherein said chemotherapeutic agent is selected from the group consisting of: cisplatin, carboplatin, pemetrexed and gemcitabine.

17. The method of claim 16, wherein said chemotherapeutic agent comprises cisplatin and pemetrexed.

18. The method of claim 15, further comprising administering to said human a COX-2 inhibitor.

19. The method of claim 18, wherein said COX-2 inhibitor comprises celecoxib.

20. The method of claim 12, wherein said mesothelium comprises mesothelium surrounding ovary and wherein said cancer comprises ovarian cancer, and wherein said transgene codes for a vascular endothelial growth factor or active fragment thereof.

21. In a method of treating organ cancer in a human by administering to said human recombinant virus comprising a homomimetic transgene, the improvement comprising administering to said human an agent selected from the group consisting of: cisplatin, carboplatin, pemetrexed and gemcitabine.

22. The method of claim 21, wherein said organ is located in a mesothelium-lined body cavity.

23. The method of claim 22, wherein said organ is selected from the group consisting of: lung, kidney, adrenal gland, ovary, prostate, pancreas and bladder.

24. The method of claim 23, wherein said organ comprises lung and wherein said cancer comprises malignant pleural mesothelioma.

25. The method of claim 21, wherein said administering recombinant virus to said human comprises irrigating at least part of mesothelium lining a body cavity of said human with a solution comprising said recombinant virus.

26. The method of claim 21, wherein said homeomimetic transgene codes for interferon, and wherein said interferon comprises interferon alpha 2b.

27. The method of claim 21, wherein said chemotherapeutic comprises cisplatin and pemetrexed.

28. The method of claim 21, further comprising administering to said human a COX-2 inhibitor.

29. The method of claim 28, wherein said COX-2 inhibitor comprises celecoxib.

30. In a method of treating cancer in a human by administering a chemotherapeutic agent to said human, the improvement comprising further administering to said human recombinant virus comprising a homomimetic transgene, and further administering to said human agent which is a COX-2 inhibitor

31. (canceled)

32. (canceled)

33. (canceled)

34. (canceled)

35. (canceled)

36. (canceled)

37. (canceled)

38. (canceled)

39. (canceled)

40. (canceled)

41. (canceled)
